SEQUENCE LISTING (9 sequences)

SEQ ID NO: 1 - DNA, 432 bp, Homo sapiens
tagtgccctggagagcatggggagacccgggaccctgctgggtttctctgtcacaaaggaaaataatccccctggtgtgacagacccaaggacagaacacagcagaggtcagcactggggaagacaggttgtcctcccaggggatgggggtccatccaccttgccgaaaagatttgtctgaggaactgaaaatagaagggaaaaaagaggagggacaaaagaggcagaaatgagaggggaggggacagaggacacctgaataaagaccacacccatgacccacgtgatgctgagaagtactcctgccctaggaagagactcagggcagagggaggaaggacagcagaccagacagtcacagcagccttgacaaaacgttcctggaactcaagctcttctccacagaggaggacagagcagacagcagagacc

SEQ ID NO: 2 - protein, 876 aa, Homo sapiens
MRQGAPARGCRWFVVWALLGLTLGVLVASAAPSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKV

SEQ ID NO: 3 - DNA, 277 bp, Homo sapiens
ttaccgaccacccgcaaacagcagggtcccctgggcttcccaagccgcgcacctctccgccccgcccctgcgccctccttcctcgcgtctgcccctctcccccaccccgccttctccctccccgccccagcggcgcatgcgccgcgctcggagcgtgtttttataaaagtccggccgcggccagaaacttcagtttgttggctgcggcagcaggtagcaaagtgacgccgagggcctgagtgctccagtagccaccgcatctggagaaccagcggtt

SEQ ID NO: 4 - protein, 554 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 19 aa, Homo sapiens
ATNFSLLKQAGDVEENPGP

SEQ ID NO: 7 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 8 - DNA, 20 bp, Homo sapiens
ggcattcaccgcgtgcctta

SEQ ID NO: 9 - DNA, 22 bp, Homo sapiens
tgagctacagtgcttcatctca

(19) United States
(12) Patent Application Publication
(10) Pub. No.: US 2026/0091069 A1
(43) Pub. Date: Apr. 2, 2026

(54) TRANSCRIPTIONAL AND TRANSLATIONAL DUAL REGULATED ONCOLYTIC HERPES SIMPLEX VIRUS VECTORS

(71) Applicant: VIROGIN BIOTECH CANADA LTD, Richmond (CA)

(72) Inventors: William Wei-Guo Jia, Vancouver (CA); Dmitry V. Chouljenko, Vancouver (CA); I-Fang Lee, Vancouver (CA); Yanal M. Murad, Vancouver (CA); Xiaohu Liu, Vancouver (CA); Guoyu Liu, Vancouver (CA); Xuexian Bu, Vancouver (BC); Zahid Delwar, Vancouver (CA); Jun Ding, Vancouver (CA)

(21) Appl. No.: 18/283,654

(22) PCT Filed: Mar. 24, 2022
(86) PCT No.: PCT/US2022/021798

§ 371 Date: Sep. 22, 2023

Related U.S. Application Data
(60) Provisional application No. 63/165,667, filed on Mar. 24, 2021.
(60) Provisional application No. 63/302,481, filed on Jan. 24, 2022.

(51) Int. Cl. A61K 35/763; A61K 38/20; A61P 35/00; C12N 7/00

(52) U.S. Cl. CPC A61K 35/763; A61K 38/208; A61K 38/2086; A61P 35/00; C12N 7/00; C12N 2710/16032; C12N 2710/16071

(57) ABSTRACT

A herpes virus vector is provided with both transcriptional and translational control. Within various embodiments the herpes virus vector is based upon a modified herpes virus and has both ICP27 and ICP34.5 under control of a CEA promoter and miRNA-124/143, respectively, and deletion of at least one copy of terminal repeat long region is provided to increase safety without sacrificing efficacy. The herpes virus vector can also incorporate a virus-expressed cytokine cassette encoding IL-12, IL-15/IL-15RA under the control of CXCR4 promoter.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### FIELD OF THE INVENTION

The present invention relates generally to oncolytic herpes simplex virus (oHSV) vectors that express molecules that stimulate the immune system

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “VIRO413_ST25.txt,” a creation date of Jan. 24 2022, and a size of 17.7 KB. The Sequence Listing filed via EFS-Web is part of the specification and is incorporated in its entirety by reference herein

### BACKGROUND

Malignant tumors are a serious threat to human life and health. Although a variety of standard treatment options exist, such as surgery, radiotherapy, chemotherapy, targeted therapy, and immunotherapy (including immune checkpoint inhibitors), most patients with advanced tumors still have poor prognosis. At present, tumor immunotherapy has made breakthrough progress in the treatment of tumors. Immune-targeted drug therapy (e.g., immune checkpoint suppression) and immune cell therapy (e.g., chimeric antigen receptor T-cell (CAR-T)) have triggered changes in the field of anti-tumor therapy. However, among the currently approved indications for checkpoint inhibitors, the single-drug effective rate is only about 30% (Jiang et al., 2020, Progress and Challenges in Precise Treatment of Tumors With PD-1/L1 Blockade. Frontiers in Immunology, 11 (March)); while CAR-T therapy mainly only targets Cluster of Differentiation 19 (CD19) and B cell maturation antigen (BCMA) that are highly expressed by B cell tumors. The clinical effectiveness in solid tumors has yet to be confirmed (Long et al., 2018, CAR T Cell Therapy of Non-hematopoietic Malignancies: Detours on the Road to Clinical Success. Frontiers in Immunology, 9 (December), 2740). There are still many malignant tumors where there is clear long-term evidence as to the benefits of immunotherapy.

There is no clinically effective treatment for malignant tumors relapsed after and refractory to standard treatment, and patients with this condition are likely to die sooner due to the extensive tumor metastasis or invasion of important organs. Therefore, these patients have an extremely high unmet need for effective treatment, leading to an urgent need to develop new treatment methods to control the progression of the disease and prolong the survival of patients.

The present invention overcomes shortcomings of current cancer therapies, including immunotherapies, and further provides additional unexpected benefits.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, the invention relates to compositions and methods for treating cancer with recombinant herpes virus vectors. Within preferred embodiments of the invention, the recombinant vectors are controlled both transcriptionally and post-transcriptionally (translationally) in order to provide more precise control of the oncolytic potential of the virus.

Within one embodiment of the invention recombinant herpes viruses are provided comprising a modified oncolytic herpes virus genome, wherein the modified herpes virus genome comprises at least one miRNA target sequence operably linked to a first copy of an ICP34.5 gene, and a second copy of the ICP34.5 gene comprises an inactivating mutation. Within various embodiments, the recombinant virus can comprise one, two, three, four, five, six, seven, eight, nine, or, ten miRNA target sequences operably linked to the first copy of the ICP34.5 gene.

Within further embodiments, the miRNA target sequences can bind at least two different miRNAs (e.g., one or more of miR-124, miR-124*, and miR-143).

Within yet other embodiments, the recombinant herpes virus can further comprise at least one nucleic acid encoding a non-viral protein. Examples of non-viral proteins include immunostimulatory factors, antibodies, and checkpoint blocking peptides, wherein the at least one nucleic acid is operably linked to a tumor-specific promoter. Within particularly preferred embodiments, the non-viral protein is one, or all of IL12, IL15, IL15 receptor alpha subunit.

Within yet other embodiments the recombinant herpes simplex virus further comprises a nucleic acid sequence encoding a glycoprotein with enhanced fusogenicity (as compared to a similar wild-type virus). Examples include a wide variety of transgenes (e.g., a fusogenic glycoprotein from Gibbon Ape Leukemia Virus “GALV”), and/or mutations which enhance HSV fusion, including for example, truncations or mutations in glycoprotein B, glycoprotein K, and or UL20.

Also provided are therapeutic compositions comprising the recombinant herpes viruses described herein, as well as methods of lysing tumor cells, and, methods of treating cancers in a subject comprising the step of administering one of the recombinant herpes viruses described herein to a subject.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

The present invention may be understood more readily by reference to the following detailed description of preferred embodiments of the invention and the Examples included herein.

FIG. 1 diagrammatically depicts the overall structural organization of the double-stranded deoxyribonucleic acid (DNA) elements of VG2025.

FIG. 2 diagrammatically depicts a Transcription and Translation Dual Regulated (“TTDR”) system.

FIG. 3 shows the results of an experiment wherein multi-nucleated fusogenic plaques are observed in VG2025 infected A549 (tumor) cells but not in MRC-5 (non-tumor) cells.

FIGS. 4A and 4B graphically shows CEA expression in different tumor cell lines, which correlates with virus copy number after infection with VG2025.

FIG. 5 graphically shows miR124/143 regulation of ICP34.5.

FIGS. 6A and 6B show the anti-tumor activity of hVG2025, measured as cell viability on 11 human tumor cell lines (FIG. 6A) and 6 mouse tumor cell lines (FIG. 6B) by in vitro culture.

FIGS. 7A and 7B graphically shows the growth curve of the two viruses in the two tumor cell lines (A549 and BxPC-3, respectively).

FIGS. 8A and 8B graphically shows payload expression of IL-12 (FIG. 8A) and IL-15 (FIG. 8B).

FIGS. 9A and 9B graphically shows payload expression from cells infected with hVG2025, VG1905 or no virus.

FIG. 10 graphically shows payload bioactivity on human IFN-g production from PHA-stimulated lymphoblasts.

FIGS. 11A, 11B, 11C, 11D, 11E, 1F and 11G graphically show A549 Tumor size after hVG2025 treatment.

FIG. 12 graphically shows changes in body weight after hVG2025 treatment.

FIGS. 13A, 13B, 13C, 13D, 13E, 13F, and 13G graphically shows the size of BxPC3 tumors after hVG2025 treatment.

FIG. 14 graphically shows BxPC3 tumor model body weight after hVG2025 treatment.

FIGS. 15A, 15B, 15C, 15D, 15E and 15F graphically show a comparison of hVG2025 and 34.5 (−) HSV-1 on tumor size.

FIG. 16 graphically shows the body weight of DBA/2 mice that were subcutaneously injected with hVG2025.

FIG. 17 graphically shows the percent survival of DBA/2 mice that were subcutaneously injected with hVG2025.

FIG. 18 graphically shows the body weight of DBA/2 mice that were nasally inoculated with hVG2025.

FIG. 19 graphically shows the percent survival of DBA/2 mice that were nasally inoculated with hVG2025.

FIG. 20A are photos which provides clinical observations of virus-induced symptoms.

FIG. 20B graphically depicts a survival curve.

FIG. 21 graphically provides an RNA-seq analysis of olfactory bulb and trigeminal ganglion after corneal scarification.

FIG. 22 graphically shows the sensitivity of hVG2025 to Ganciclovir.

FIG. 23 graphically shows the stability of hVG2025 at 4° C. and −80° C. for up to 1 month.

FIG. 24A graphically shows body weight changes in Hep 3B-luc burdened BALB/c nude mice treated with VG2025; FIG. 24B shows a tumor bioluminescence trace after administering VG2025; FIG. 24C provides a survival curve for treated mice; and FIG. 24D shows metastasis rates.

FIG. 25A graphically shows body weight changes in A20-luc B cell lymphoma burdened BALB/c nude mice treated with mVG2025; FIG. 25B shows a tumor bioluminescence trace after administering mVG2025; and FIG. 25C shows metastasis rates.

FIG. 26A graphically shows body weight changes in A20-luc B cell lymphoma burdened BALB/c nude mice treated with mVG2025; FIG. 26B shows a tumor bioluminescence trace after administering mVG2025; and FIG. 26C shows metastasis rates.

FIGS. 27A and 27B graphically depict average tumor volumes of treated tumor and untreated contralateral tumor after treatment up to day 9 post treatment initiation.

FIGS. 28A, 28B, 28C and 28D graphically depicts individual tumor size in treated tumors and contralateral tumors.

FIG. 29 graphically depicts a survival curve.

FIGS. 30A and 30B graphically depict average tumor volumes of treated tumor and untreated contralateral tumor after treatment up to day 17 post treatment initiation.

FIGS. 31A, 31B, 31C and 31D graphically depict individual tumor size before and after subcutaneous tumor reimplantation.

FIG. 32 graphically depicts a survival curve.

FIGS. 33A and 33B graphically depicts a plot of human IL-12p70, and IL-15/IL-15Ralpha complex in tumors.

FIGS. 34A and 34B graphically depicts a plot of human IL-12p70, and IL-15/IL-15Ralpha complex in serum.

### DETAILED DESCRIPTION OF THE INVENTION

As noted above, the present invention provides recombinant herpes virus vectors which are controlled both transcriptionally and post-transcriptionally (translationally) in order to provide more precise control of the oncolytic potential of the virus.

In order to further an understanding of the various embodiments herein, the following sections are provided which describe various embodiments: A. Oncolytic Herpes Viruses; B. Specific Herpes Virus Constructs-VG2025; C. Therapeutic Compositions, and D. Administration

### A. Oncolytic Herpes Viruses

Briefly, Herpes Simplex Virus (HSV) 1 and 2 are members of the Herpesviridae family, which infects humans. The HSV genome contains two unique regions, which are designated unique long (UL) and unique short (Us) region. Each of these regions is flanked by a pair of inverted repeat sequences. There are about 75 known open reading frames. The viral genome has been engineered to develop oncolytic viruses for use in e.g. cancer therapy. Tumor-selective replication of HSV may be conferred by mutation of the HSV ICP34.5 (also called γ34.5) gene. HSV contains two copies of ICP34.5. Mutants inactivating one or both copies of the ICP34.5 gene are known to lack neurovirulence, i.e. be avirulent/non-neurovirulent and be oncolytic. Tumor selective replication of HSV may also be conferred by controlling expression of key viral genes such as ICP27 and/or ICP4.

The term “oncolytic herpes virus” or “OHV” refers generally to a herpes virus capable of replicating in and killing tumor cells. The term “oncolytic herpes simplex virus” or “oHSV” refers to a herpes simplex virus that is capable of replicating in and killing tumor cells.

Suitable oncolytic HSV may be derived from either HSV-1 or HSV-2, including any laboratory strain or clinical isolate. In some embodiments, the oHSV may be derived from one of laboratory strains HSV-1 strain 17, HSV-1 strain F, or HSV-2 strain HG52. In other embodiments, it may be derived from non-laboratory strain JS-1. Other suitable HSV-1 viruses include HrrR3 (Goldstein and Weller, J. Virol. 62, 196-205, 1988), G207 (Mineta et al. Nature Medicine. 1 (9): 938-943, 1995; Kooby et al. The FASEB Journal, 13 (11): 1325-1334, 1999); G47Delta (Todo et al. Proceedings of the National Academy of Sciences. 2001; 98 (11): 6396-6401); HSV 1716 (Mace et al. Head & Neck, 2008; 30 (8): 1045-1051; Harrow et al. Gene Therapy. 2004; 11 (22): 1648-1658); HF10 (Nakao et al. Cancer Gene Therapy. 2011; 18 (3): 167-175); NV1020 (Fong et al. Molecular Therapy, 2009; 17 (2): 389-394); T-VEC (Andtbacka et al. Journal of Clinical Oncology, 2015: 33 (25): 2780-8); J100 (Gaston et al. PloS one, 2013; 8 (11): e81768); M002 (Parker et al. Proceedings of the National Academy of Sciences, 2000; 97 (5): 2208-2213); NV1042 (Passer et al. Cancer Gene Therapy. 2013; 20 (1): 17-24); G207-IL2 (Carew et al. Molecular Therapy, 2001; 4 (3): 250-256); rQNestin34.5 (Kambara et al. Cancer Research, 2005; 65 (7): 2832-2839); G47Δ-mIL-18 (Fukuhara et al. Cancer Research, 2005; 65 (23): 10663-10668); and those vectors which are disclosed in PCT applications PCT/US2017/030308 entitled “HSV Vectors with Enhanced Replication in Cancer Cells”, and PCT/US2017/018539 entitled “Compositions and Methods of Using Stat1/3 Inhibitors with Oncolytic Herpes Virus”, all of the above of which are incorporated by reference in their entirety.

Other representative examples of oncolytic herpes viruses are described in U.S. Pat. Nos. 7,223,593, 7,537,924, 7,063,835, 7,063,851, 7,118,755, 8,216,564, 8,277,818, and 8,680,068, all of which are incorporated by reference in their entirety.

The oHSV vector has at least one γ34.5 gene that is modified with miRNA target sequences in its 3′ UTR as disclosed herein; there are no unmodified γ34.5 genes in the vector. In some embodiments, the oHSV has two modified γ34.5 genes; in other embodiments, the oHSV has only one γ34.5 gene, and it is modified. In some embodiments, the modified γ34.5 gene(s) are constructed in vitro and inserted into the oHSV vector as replacements for the viral gene(s). When the modified γ34.5 gene is a replacement of only one γ34.5 gene, the other γ34.5 is deleted. Either native γ34.5 gene can be deleted. In one embodiment, the terminal repeat region, which comprises γ34.5 gene and ICP4 gene, is deleted. As discussed herein, the modified γ34.5 gene may comprise additional changes, such as having an exogenous promoter.

The oHSV may have additional mutations, which may include disabling mutations e.g., deletions, substitutions, insertions), which may affect the virulence of the virus or its ability to replicate. For example, mutations may be made in any one or more of ICP6, ICPO, ICP4, ICP27, ICP47, ICP24, ICP56. Preferably, a mutation in one of these genes (optionally in both copies of the gene where appropriate) leads to an inability (or reduction of the ability) of the HSV to express the corresponding functional polypeptide. In some embodiments, the promoter of a viral gene may be substituted with a promoter that is selectively active in target cells or inducible upon delivery of an inducer or inducible upon a cellular event or particular environment.

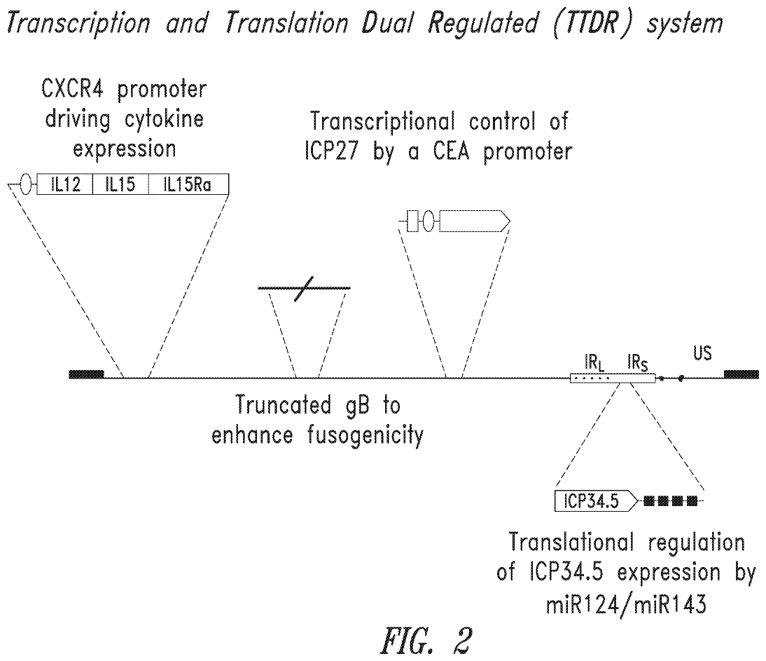

In certain embodiments the expression of ICP4 or ICP27 is controlled by an exogenous promoter, e.g., a tumor-specific promoter. Exemplary tumor-specific promoters include survivin, CEA, CXCR4, PSA, ARR2PB, or telomerase; other suitable tumor-specific promoters may be specific to a single tumor type and are known in the art. Other elements may be present. In some cases, an enhancer such as NFkB/oct4/sox2 enhancer is present. As well, the 5′UTR may be exogenous, such as a 5′UTR from growth factor genes such as FGF. See FIG. 2 for an exemplary construct.

The oHSV may also have genes and nucleotide sequences that are non-HSV in origin. For example, a sequence that encodes a prodrug, a sequence that encodes a cytokine or other immune stimulating factor, a tumor-specific promoter, an inducible promoter, an enhancer, a sequence homologous to a host cell, among others may be in the oHSV genome. Exemplary sequences encode IL12, IL15, IL15 receptor alpha subunit, OX40L, PD-L1 blocker or a PD-1 blocker. For sequences that encode a product, they are operatively linked to a promoter sequence and other regulatory sequences (e.g., enhancer, polyadenylation signal sequence) necessary or desirable for expression.

The regulatory region of viral genes may be modified to comprise response elements that affect expression. Exemplary response elements include response elements for NF-κB, Oct-3/4-SOX2, enhancers, silencers, cAMP response elements, CAAT enhancer binding sequences, and insulators. Other response elements may also be included. A viral promoter may be replaced with a different promoter. The choice of the promoter will depend upon a number of factors, such as the proposed use of the HSV vector, treatment of the patient, disease state or condition, and ease of applying an inducer (for an inducible promoter). For treatment of cancer, generally when a promoter is replaced it will be with a cell-specific or tissue-specific or tumor-specific promoter. Tumor-specific, cell-specific and tissue-specific promoters are known in the art. Other gene elements may be modified as well. For example, the 5′ UTR of the viral gene may be replaced with an exogenous UTR.

### B. Specific Herpes Virus Constructs—VG2025

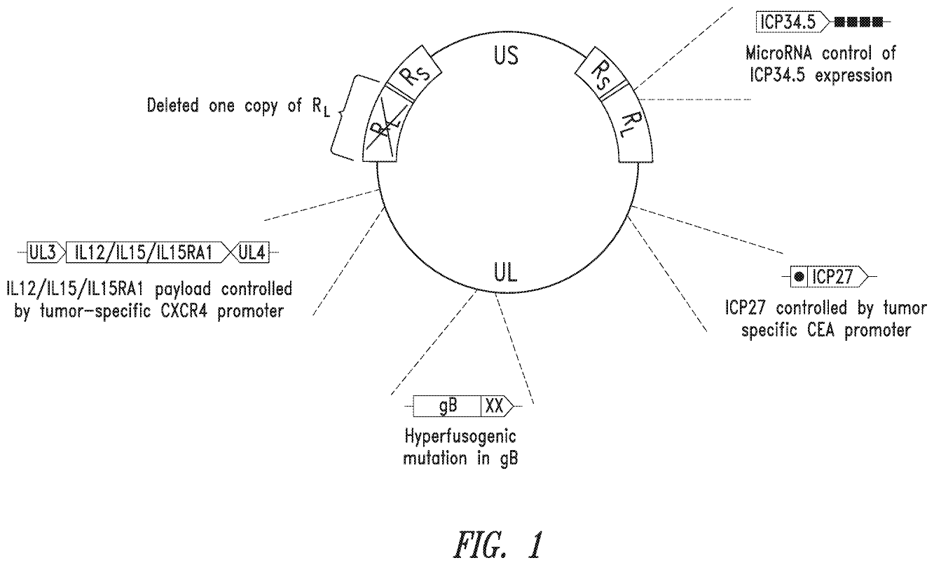

One preferred construct of the invention is provided in FIG. 1. Briefly, FIG. 1 diagrammatically depicts the overall structural organization of the double-stranded deoxyribonucleic acid (DNA) elements of VG2025. “CEA” means carcinoembryonic antigen; “CXCR4” means C-X-C Motif Chemokine Receptor 4; “gB” means glycoprotein B; “ICP” means infected cell polypeptide; “IL” means interleukin; “R_{L}” means repeat long; “RNA” means ribonucleic acid; “miR” means microRNA; “R_{S}” means repeat short; “UL” means unique long; “US” means unique short.

VG2025 is a recombinant HSV-1 platform that utilizes both transcriptional and translational dual-regulation (“TTDR”—see FIG. 2) of key viral genes to limit virus replication to tumor cells and enhance tumor-specific virulence without compromising safety. In addition, VG2025 expresses a payload cassette composed of IL12, IL15 and IL15 receptor alpha subunit. The payload expression is controlled by a CXCR4 promoter for tumor specific immune stimulation. Finally, the viral glycoprotein B (gB) in VG2025 was truncated to facilitate virus spread in the tumor microenvironment by enhanced fusogenicity.

### 1. Post-Transcriptional (Translational) Regulation

In VG2025, ICP34.5 expression is post-transcriptionally (translationally) regulated. Briefly, in wild-type HSV-1, there are 2 copies of the ICP34.5 gene. In VG2025, one copy of ICP34.5 has been deleted. For the remaining ICP34.5 gene, VG2025 inserts multiple copies of binding domains for miR124 and miR143 in the 3′UTR region to regulate its expression post-transcriptionally.

ICP34.5 is encoded by the HSV late gene g-34.5. It is well known for its function of suppressing anti-viral immunity of host cells, particularly neuronal cells, to cause neurotoxicity. To abolish the functions of ICP34.5 in neurons and other normal cells while retaining its activity in tumor cells for robust replication, instead of deleting the gene or using a specific promoter to control the expression of ICP34.5 to target gliomas, VG2025 uses microRNAs as a post-transcriptional control to achieve differential expression of ICP34.5 in tumor cells. Briefly, micro RNAs (also referred to as “miRNA” s or “miR” s) are ˜22 nucleotides, noncoding small RNAs coded by miRNA genes, which are transcribed by RNA polymerase Il to produce primary miRNA (pri-miRNA). Mature single-stranded (ss) miRNA forms the miRNA-associated RNA-induced silencing complex (miRISC). miRNA in miRISC may influence gene expression by binding to the 3′-untranslated region (3′-UTR) in the target mRNA. This region consists of sequences recognized by miRNA. If the complementarity of the miRNA: mRNA complex is perfect, the mRNA is degraded by Ago2, a protein belonging to the Argonaute family. However, if the complementarity is not perfect, the translation of the target mRNA is not fully degraded, but is suppressed.

MiRNAs are expressed differentially in a tissue specific fashion. One of the examples is miR124. While the precursors of miR-124 from different species are different, the sequences of mature miR-124 in human, mice, rats are completely identical. MIR-124 is the most abundantly expressed miRNA in neuronal cells and is highly expressed in the immune cells and organs (Qin et al., 2016, miRNA-124 in immune system and immune disorders. Frontiers in Immunology, 7 (OCT), 1-8). Another example of differential expression of miRNA is miR143 (Lagos-Quintana et al., 2002, Identification of tissue-specific MicroRNAs from mouse. Current Biology, 12 (9), 735-739. MiR-143 is constitutively expressed in normal tissues but significantly downregulated in cancer cells (Michael et al., 2003, Reduced Accumulation of Specific MicroRNAs in Colorectal Neoplasia. Molecular Cancer Research, 1 (12), 882-892. A representative example of the nucleic acid sequence of miR-124 is as set forth in SEQ ID NO: 8, and an example of the nucleic acid sequence of miR-143 is as set forth in SEQ ID NO: 9.

The 3′ UTR region of ICP34.5 gene in VG2025 contains multiple copies of binding domains (also referred to as “miRNA target sequences”, “miRNA binding sequences” or “miRNA binding sites”) that are completely complementary to miR124 and miR143. Binding of miR124 and miR143 to the 3′UTR of ICP34.5 mRNA causes degradation of the mRNA; therefore the gene is post-transcriptionally downregulated in normal cells but not tumor cells. This design allows differential expression of ICP34.5 in tumor cells.

### 2. Expression of ICP27 in VG2025 is Transcriptionally Controlled

HSV-1 viral replication depends on a cascade of expression of viral genes, with immediate early gene products (particularly ICP4 and ICP27) controlling subsequent expression of viral early genes and late genes that govern the lytic replication cycle of the virus. Deletion of ICP4 or ICP27 results in complete abrogation of viral replication and a significant reduction in viral gene expression, which makes ICP4 and ICP27 excellent targets for tumor specific regulation in oncolytic HSV.

While ICP4 is a major transcription factor regulating viral gene expression, ICP27 is a multi-functional protein that regulates transcription of many virus genes. ICP27 functions in all stages of mRNA biogenesis from transcription, RNA processing and export through to translation. ICP27 has also been implicated in nuclear protein quality control, cell cycle control, activation of stress signaling pathways and prevention of apoptosis.

In VG2025, the native promoter of ICP27 is replaced with a 432 bp promoter for human carcinoembryonic antigen (CEA) (Beauchemin and Arabzadeh, 2013, Carcinoembryonic antigen-related cell adhesion molecules (CEACAMs) in cancer progression and metastasis. Cancer and Metastasis Reviews, 32 (3-4), 643-671; Hammarström 1999, The carcinoembryonic antigent (CEA) family. Structures, suggested functions and expression in normal and malignant tissues. In Seminars in cancer biology 9 (2), pp. 67-81; Kodera et al. 1993, Expression of carcinoembryonic antigen (CEA) and nonspecific crossreacting antigen (NCA) in gastrointestinal cancer; the correction with degrees of differentiation. In Br. J Cancer 68 (1), pp 130-136). CEA belongs to a sub-group of 12 genes called carcinoembryonic antigen cell adhesion molecules (CEACAMs) as a part of 22 gene family (Beauchemin & Arabzadeh, 2013). CEA plays significant roles in cellular processes including inhibition of differentiation programs, inhibition of anoikis and apoptosis, and disruption of cell polarization and tissue architecture (Beauchemin & Arabzadeh, 2013). For example, the nucleic acid sequence of CEA promoter is as set forth in SEQ ID NO: 1.

### 3. Payload Expression of VG2025 is Tumor-Enhanced

VG2025 co-expresses IL12, IL15 and IL15 receptor alpha subunit to further stimulate an immunomodulatory response. Expression of IL12 promotes polarization of antigen exposed T cells towards an inflammatory and anti-tumor T_{H}1 phenotype, while IL-15 activates NK cells to further increase tumor killing and activation of antigen presenting cells. In addition to IL15 expression, VG2025 encodes IL15Rα to further enhance immune stimulation. For example, the human IL12 can comprise the amino acid sequence as set forth in SEQ ID NO: 4; the human IL15 can comprise the amino acid sequence as set forth in SEQ ID NO: 5; and the human IL15 receptor alpha subunit can comprise the amino acid sequence as set forth in SEQ ID NO: 7.

Transcription of IL-12, IL-15, and IL-15Ra is driven by the single promoter (CXCR4) and the polypeptides are linked with 2A self-cleaving peptides (Z. Liu et al., 2017, Systematic comparison of 2A peptides for cloning multi-genes in polycistronic vector. Scientific Reports, 7 (2), 1-9) that generate the 3 individual proteins through a mechanism of ribosomal skipping during translation. For example, the 2A peptide can comprise the amino acid sequence as set forth in SEQ ID NO: 6. While the payloads are meant to be expressed intratumorally, unwanted expression in normal tissues may occur in case the virus “leaks” to extratumor area or when the virus is delivered systemically. To mitigate potential risk of IL12/15 expression outside of tumor bed, the expression cassette of the payloads in VG2025 is driven by a single promoter for CXC chemokine receptor 4 (CXCR4) (Moriuchi et al., 1997, Cloning and analysis of the promoter region of CXCR4, a coreceptor for HIV-1 entry. Journal of Immunology (Baltimore, Md.: 1950), 159 (9), 4322-429; Caruz et al., 1998, Genomic organization and promoter characterization of human CXCR4 gene. FEBS Letters, 426 (2), 271-278. CXCR4 is a seven-transmembrane G protein-coupled receptor that was originally isolated from human blood monocytes as a cofactor for HIV virus fusion and entry of T cells (Moriuchi et al. 1997). For example, the nucleic acid sequence of CXCR4 promoter is as set forth in SEQ ID NO: 3.

Representative examples of selected expression cassettes/vectors are also described in PCT Publication WO 2018/026872, which is incorporated by reference in its entirety.

### 4. Truncated Glycoprotein B (GB)

HSV-1 membrane fusion is a crucial step of infection. It is dependent on four essential viral glycoproteins (gB, gD, gH, and gL), which mediate entry into host cells by merging the viral envelope with a host cell membrane. The core fusion protein is glycoprotein B (gB), a 904-residue glycosylated transmembrane protein encoded by the UL27 gene of HSV-1. Multiple types of mutations within the cytoplasmic domain of gB have yielded a hyperfusogenic phenotype, increasing cell-cell fusion (Chowdary & Heldwein, 2010, Synctial Phenotype of C-Terminally Truncated Herpes Simplex Virus Type 1 gB is Associated with Diminished Membrane Interactions. Journal of Virology, 84 (10), 4923-4935. In one embodiment, gB may be modified by truncating C-terminal amino acids 877 to 904 from the full-length protein. For example, the amino acid sequence of the truncated glycoprotein B is as set forth in SEQ ID NO: 2.

### 5. Summary

VG2025 is an oncolytic virus product with ICP27 and ICP34.5 under control of CEA promoter and miRNA-124/143, respectively. hVG2025 also incorporates a virus-expressed cytokine cassette encoding IL-12, IL-15/IL-15RA under the control of CXCR4 promoter. The expression control mechanisms in VG2025 are designed to increase safety without sacrificing efficacy. Specific modifications to wild type-HSV-1 strain 17+ are set forth below in Table 1.

TABLE 1
Genetic Modification in VG2025 from wild type HSV-1, strain 17+

| | Modification | Modification | |
|---|---|---|---|
| Modification | Type | Location | Function |
| Deletion of terminal | Deletion | Terminal repeat | Wild type HSV-1 contains two |
| repeat long (TR_{L}) | | long (TR_{L}) inverted | identical copies of a long inverted |
| containing ICP0, and | | repeat | repeat R_{L} designated as TR_{L} and IR_{L} |
| ICP34.5 genes | | | and two identical copies of a short |
| | | | inverted repeat R_{S} designated as |
| | | | TR_{S} and IR_{S}. Removal of one copy |
| | | | may attenuate virulence. |
| Replacement of native | Replacement | ICP27 gene | To facilitate the virus replication in |
| ICP27 promoter with | | promoter | CEA positive tumor cells |
| (CEA) promoter | | | |
| Insertion of binding | Insertion | ICP34.5 gene 3′-UTR | To inhibit expression of ICP34.5 in |
| sites for miR143 and | | | cells with high expression of |
| miR124 in the ICP34.5 | | | miR143 and/or miR124 |
| 3′-UTR | | | |
| Deletion of 84 bp in 3′ | Deletion | 3′ end of (gB) coding | To make HSV-1 gB protein |
| end of glycoprotein B | | region | truncated by 28 amino acids at its |
| (gB) coding region | | | c-terminus for enhanced |
| | | | fusogenicity |
| Insertion of expression | Insertion | Between UL3 and | To make the virus express IL-12, |
| cassette of IL-12, IL-15, | | UL4 genes | IL-15, and IL-15Rα in CXCR4 |
| and IL-15Rα under | | | positive tumor cells |
| CXCR4 promoter | | | |

CEA = carcinoembryonic antigen;
CXCR4 = C-X-C Motif Chemokine Receptor 4;
gB = glycoprotein B;
HSV-1 = herpes simplex virus-1;
ICP27 = infected cell polypeptide 27;
ICP34.5 = infected cell polypeptide 34.5;
IL = interleukin;
miR = microRNA;
Rα = receptor alpha;
TR_{L} = terminal repeat long;
IR_{L} = internal repeat long;
TR_{S} = terminal repeat short;
IR_{S} = internal repeat short;
UL = unique long

VG2025 is a conditionally replicating oncolytic HSV-1 virus. The genome of VG2025 deleted the terminal repeat long (TRL) sequence of HSV-1 that contains one copy of ICP34.5, ICPO and LAT. The remaining copy of ICP34.5 has an insertion in its 3′UTR region containing multiple copies of binding domains for miRNA miR-124 and miR-143, which are highly expressed in neurons and normal tissues but not in tumor cells. The product is further modified by replacing the native viral promoter for the essential viral gene UL54 which encodes ICP27 (infected cell polypeptide 27) with a tumor-specific promoter from human carcinoembryonic antigen (CEA) gene. VG2025 also expresses a potent immunomodulatory payload, consisting of IL-12, IL-15, and IL-15Ra, which is controlled by a tumor selective C-X-C Motif Chemokine Receptor 4 (CXCR4) promoter. Finally, VG2025 has a glycoprotein B (gB) truncation to enhance fusogenic activity, to facilitate virus spread within the tumor microenvironment. mVG2025 and hVG2025 are very similar, except that hVG2025 has a human form of IL-12, versus a murine form in mVG2025.

As described in more detail below, preclinical pharmacology studies of VG2025 have shown significant anti-cancer activity in BxPC3 pancreatic cancer, Hep 3B liver cancer, A20 B-cell lymphoma, CT26 colon cancer, and A549 NSCLC-tumor-bearing mouse models.

### C. Therapeutic Compositions

Therapeutic compositions are provided that may be used to prevent, treat, or ameliorate the effects of a disease, such as, for example, cancer. More particularly, therapeutic compositions are provided comprising at least one oncolytic virus as described herein.

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus and that is not toxic to the subject to whom it is administered (see generally Remington: The Science and Practice of Pharmacy, Lippincott Williams & Wilkins; 21st ed. (May 1, 2005 and in The United States Pharmacopeia: The National Formulary (USP 40-NF 35 and Supplements).

In the case of an oncolytic virus as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioabsorbable matrix materials, implantation elements containing the oncolytic virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethylene glycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the oHSV to a cancer cell will preferably not induce an immune response in the individual (subject) receiving the composition (and will preferably be administered without undue toxicity).

The compositions provided herein can be provided at a variety of concentrations. For example, dosages of oncolytic virus can be provided which range from about 10⁴ pfu to about 10¹⁰ pfu. Within further embodiments, the dosage can range from about 10⁶ pfu to about 10⁷ pfu, or from about 10⁷ pfu to about 10⁸ pfu, or from about 10⁸ pfu to 10⁹ pfu, and may be administered as a single dose or as multiple doses spread out over time. Doses may be administered daily, weekly, biweekly, monthly, or bimonthly, and dosage frequency may be cyclical, with each cycle comprising a repeating dosage pattern (e. g. once a week or biweekly dose administration for about 4 weeks comprising one cycle, repeating for up to about 24 cycles). Within other embodiments of the invention, the virus can be provided in ranges from about 5×10⁴ pfu/kg to about 2×10⁹ pfu/kg for intravenous delivery in humans. For intratumoral injection, the preferred dosage can range from about 10⁶ pfu to about 10⁹ pfu per dose (with an injectable volume which ranges from about 0.1 mL to about 5 mL).

Within certain embodiments of the invention, lower or higher dosages than standard may be utilized. Hence, within certain embodiments less than about 10⁶ pfu or more than about 10⁹ pfu can be administered to a patient.

The compositions may be stored at a temperature conducive to stable shelf-life and includes room temperature (about 20° C.), 4° C., −20° C., −80° C., and in liquid N2. Because compositions intended for use in vivo generally do not have preservatives, storage will generally be at colder temperatures. Compositions may be stored dry (e.g., lyophilized) or in liquid form.

### D. Administration

In addition to the compositions described herein, various methods of using such compositions to treat or ameliorate cancer are provided, comprising the step of administering an effective dose or amount of oHSV as described herein to a subject.

The terms “effective dose” and “effective amount” refers to amounts of the oncolytic virus that is sufficient to effect treatment of a targeted cancer, e.g., amounts that are effective to reduce a targeted tumor size or load, or otherwise hinder the growth rate of targeted tumor cells. More particularly, such terms refer to amounts of oncolytic virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The compositions are used to treat cancer. The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemia's, lymphomas, myelomas and sarcomas. Representative forms of leukemias include acute myeloid leukemia (AML) and representative forms of lymphoma include B cell lymphomas. Further examples include, but are not limited to cancer of the bile duct, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemias and lymphomas), kidney, larynx, lung, liver, oral cavity, ovaries, pancreas, prostate, skin (e.g., melanoma and squamous cell carcinoma), GI (e.g., esophagus, stomach, and colon) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells).

Within certain embodiments of the invention the cancer can be resistant to or refractory from conventional treatment (e.g. conventional chemotherapy and/or radiation therapy). Benign tumors and other conditions of unwanted cell proliferation may also be treated.

Particularly preferred cancers to be treated include those with high levels of CEA expression. Representative examples include lung tumors, breast and prostate tumors, hematopoietic cell tumors (e.g., leukemias and lymphomas), glioblastomas, tumors of the gastro-intestinal tract (and associated organs) e.g., esophagus, cholangiocarcinoma, anal, stomach, intestine, pancreatic, colon and liver, and all surface injectable tumors (e.g., melanomas).

The recombinant herpes simplex viruses described herein may be given by a route that is e.g. oral, topical, parenteral, systemic, intravenous, intramuscular, intraocular, intrathecal, intratumoral, subcutaneous, or transdermal. Within certain embodiments the oncolytic virus may be delivered by a cannula, by a catheter, or by direct injection. The site of administration may be directly into the tumor, adjacent to the tumor, or at a site distant from the tumor. The route of administration will often depend on the type of cancer being targeted.

The optimal or appropriate dosage regimen of the oncolytic virus is readily determinable within the skill of the art, by the attending physician based on patient data, patient observations, and various clinical factors, including for example a subject's size, body surface area, age, gender, and the particular oncolytic virus being administered, the time and route of administration, the type of cancer being treated, the general health of the patient, and other drug therapies to which the patient is being subjected. According to certain embodiments, treatment of a subject using the oncolytic virus described herein may be combined with additional types of therapy, such as administration of a different oncolytic virus, radiotherapy, administration of a checkpoint inhibitor, chemotherapy using, e.g., a chemotherapeutic agent such as etoposide, ifosfamide, adriamycin, vincristine, doxycycline, and others.

Recombinant herpes simplex viruses described herein may be formulated as medicaments and pharmaceutical compositions for clinical use and may be combined with a pharmaceutically acceptable carrier, diluent, excipient or adjuvant. The formulation will depend, at least in part, on the route of administration. Suitable formulations may comprise the virus and inhibitor in a sterile medium. The formulations can be fluid, gel, paste or solid forms. Formulations may be provided to a subject or medical professional.

A therapeutically effective amount is preferably administered. This is an amount that is sufficient to show benefit to the subject. The actual amount administered, and the time-course of administration will depend at least in part on the nature of the cancer, the condition of the subject, site of delivery, and other factors.

Within yet other embodiments of the invention the oncolytic virus can be administered by a variety of methods, e.g., intratumorally, intravenously, or, after surgical resection of a tumor.

### EXAMPLES

Overview: All viral mutagenesis can be performed in Escherichia coli using standard lambda Red-mediated recombineering techniques implemented on the viral genome cloned into a bacterial artificial chromosome (BAC) (see generally: Tischer B K, Smith G A, Osterrieder N. Methods Mol Biol. 2010; 634:421-30. doi: 10.1007/978-1-60761-652-8_30. PMID: 20677001; Tischer B K, von Einem J, Kaufer B, and Osterrieder N., BioTechniques 40:191-197, February 2006 (including the Supplementary Material, doi: 10.2144/000112096; and Tischer BK, Smith, GA and Osterrieder N. Chapter 30, Jeff Braman (ed.), In Vitro Mutagenesis Protocols: Third Edition, Methods in Molecular Biology, vol. 634, doi: 10.1007/978-1-60761-652-8_30, Springer Science+Business Media, LLC 2010).

BAC recombineering requires the presence of exogenous BAC DNA within the viral genome to facilitate mutagenesis in E. coli. The BAC sequence is most commonly inserted either between viral genes such as the HSV genes US1/US2, UL3/UL4 and/or UL50/UL51, or, into the thymidine kinase (TK) gene, which can disrupt expression of native TK. TK-deficient viral vectors may include an expression cassette for a copy of the native viral thymidine kinase (TK) gene under the control of a constitutive promoter inserted into a non-coding region of the viral genome. Alternatively, TK function may be restored by removing the exogenous BAC sequences via homologous recombination to reconstitute the native TK gene sequence. Presence of a functional TK gene enhances virus safety by rendering the virus sensitive to common treatment with guanosine analogues, such as ganciclovir and acyclovir.

Table of Abbreviations

| | |
|---|---|
| Akt | protein kinase B |
| Beclin1 | human beclin 1 gene |
| BCMA | B cell maturation antigen |
| BLA | Biologics License Application |
| CAR-T | chimeric antigen receptor T-cell |
| CD | Cluster of Differentiation |
| CR | Complete response |
| CXCR4 | C-X-C Motif Chemokine Receptor 4 |
| DRG | Dorsal root ganglia |
| eIF2a | eukaryotic translation initiation factor 2A |
| gB | Glycoprotein B |
| HSV-1 | herpes simplex virus-1 |
| ICP | Infected cell protein |
| IE | Infective endocarditis |
| IL | interleukin |
| IND | Investigational new drug |
| IRF | interferon regulatory factor |
| IT | intratumor |
| LAT | Latency associated transcript |
| miRNA | microRNA |
| NfκB | nuclear factor κB |
| NSCLC | non-small-cell lung cancer |
| OS | Overall survival |
| OV | Oncoviral |
| PD | Progressive disease |
| PFS | progression free survival |
| PHS | Public Health Service |
| PR | Partial response |
| RSC | rabbit skin cells |
| Rα | Receptor alpha |
| R_{L} | Repeat long |
| RNA | ribonucleic acid |
| SD | Stable disease |
| TBK1 | TANK-binding kinase |
| TME | tumor microenvironment |
| TR_{L} | terminal repeat long |
| TTDR | transcriptional and translational dual-regulation |
| UL | Unique long |
| US | Unique short |
| UTR | untranslated region |

### Example 1

### Testing Fusogenicity of VG2025

Objective: VG2025 incorporates a hyperfusogenic mutation whereby all amino acids between aa876 and the stop codon in gB are deleted. This study is to demonstrate the hyperfusogenic effect of said mutation.

Procedure: VG2025 was used to infect A549 tumor cells and MRC-5 non-tumor cells at MOfl=0.1 and incubated for 48 hours postinfection prior to imaging.

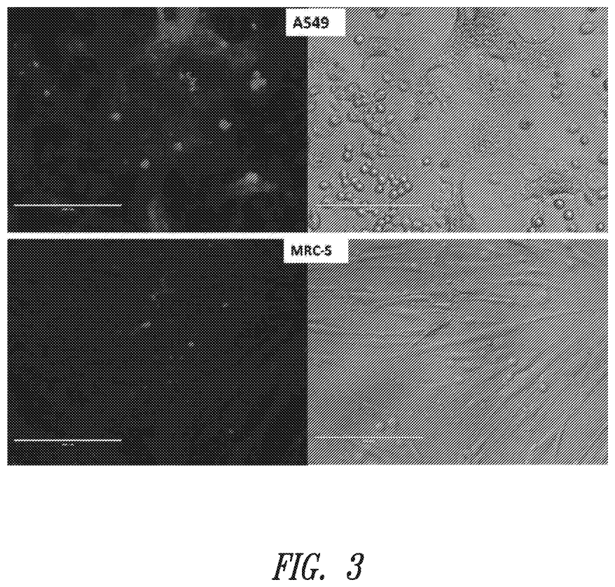

Results: As shown in FIG. 3, multi-nucleated fusogenic plaques were observed in VG2025 infected A549 cells (tumor cells) but not in MRC-5 cells (non-tumor cells).

Conclusion: VG2025 is tumor-selective and highly fusogenic.

### Example 2

Correlation of CEA Expression Level with ICP27 Expression and Viral Replication Efficiency

Objective: This study is to evaluate correlation between CEA expression level in different tumor cell lines and ICP27 expression level and virus replication efficiency of hVG2025

Procedure: The following cell lines (Table 2) were seeded into 12-well plate and incubated overnight in proper cell culture media as suggested by vendors:

TABLE 2
Cell lines screened for CEA-ICP27 correlation

| | | | CEA |
|---|---|---|---|
| No. | Cell | Source | (ng/ml) |
| 1 | A549 | human NSCLC | 107.9 |
| 2 | BxPC3 | human pancreatic cancer | 180.5 |
| 3 | U87 | human glioblastoma cancer | 0 |
| 4 | HCC2935 | human lung cancer | 207.1 |
| 5 | LS174T | human colon cancer | 154.2 |
| 6 | N87 | human stomach cancer | 109.8 |
| 7 | SW1116 | colorectal adenocarcinoma | 18 |
| 8 | SW48 | human colorectal adenocarcinoma | 32.3 |
| 9 | LOVO | human colorectal adenocarcinoma | 60.6 |
| 10 | COLO 320DM | human colorectal adenocarcinoma | 0 |
| 11 | SNU-1 | human gastric carcinoma | 0 |

VG2025 was diluted and added into cell cultures at MOI 0.1 or Mock infected. Following 2 hr incubation, the medium was changed. Cells were collected 6 hours and 18 hours after infection for RNA and DNA extraction followed by RT-qPCR to detect expression of CEA and ICP27 mRNAs. Some samples were collected at 48 hrs postinfection and processed for PCR to measure DNA copy number of ICP27. GAPDH was used to normalize each sample. Other samples of uninfected cells were used to measure CEA protein shedding in the supernatant of cell cultures using an ELISA kit (Abcam, AB99992).

Normalized Ct (ΔCt) values of CEA and ICP27 mRNA were plotted. The R and P values were calculated by regression analysis with EXCEL.

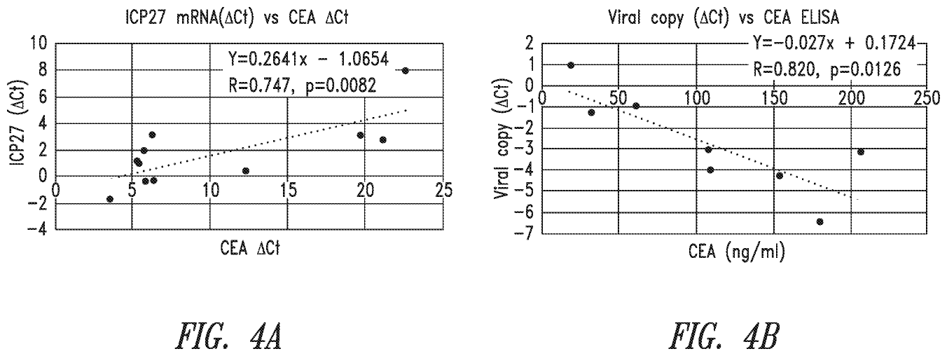

Results: Different human tumor cells express different levels of CEA. The results of this experiment are shown in FIGS. 4A and 4B. Briefly, the mRNA levels of CEA and ICP27 are expressed as ΔCt values by RT-qPCR and are plotted. The regression analysis showed ΔCt value for ICP27 mRNA positively correlates to ΔCt value of CEA mRNA, R=0.747 at p=0.0082. Virus copy numbers in hVG2025 infected cells with positive CEA by ELISA were measured as ΔCt values. The correlation between CEA protein shedding vs. virus copy number is also shown. Regression analysis showed R=0.820 at p=0.0126.

Conclusions: Certain types of tumor, including pancreatic, lung, gastrointestinal cancers, have high levels of CEA expression. Transcriptional level of ICP27 from hVG2025 showed a moderate but significant, positive correlation with transcriptional activity of CEA in some tumor cells.

CEA protein expression of tumor cells measured by the shedding in cell cultures was significantly correlated with virus copy number after infection with hVG2025.

### Example 3

miR124/143 Transcriptional Control of ICP34.5 Expression, Demonstrated by Evaluation of ICP34.5 Expression in HEK-293 Cells Transduced with miR124/143

Objectives: The objective of this study is to test the function of miRNA binding elements present in hVG2025 in controlling the expression of ICP34.5.

The viruses of the present disclosure were designed with a miR binding elements/sequences present at the 3′ UTR region of the ICP34.5 gene. In hVG2025, the targeting domains for miR regulation are miR124 and miR143. The former is highly expressed in all neuronal cells and the latter is under-expressed in most tumor cells. This will allow for using a virus with and intact ICP34.5 gene but the expression of ICP34.5 is differentially controlled via post-transcriptional regulation. While the expression of ICP34.5 will not be affected in cells and tissues not expressing the miR (tumor cells), the expression of ICP34.5 will be hampered in normal tissue expressing high levels of miRs (e.g., neuronal cells).

In this study, we used 293 FT cells, or the same cells transfected with the miR124 and miR143 mimics or a miR precursor with scrambled sequence. The cells were then superinfected with hVG2025. This will allow for the direct testing and comparison of the expression of the targeted gene in the presence or absence of the miR precursor. In addition, since the sequences of the binding domains downstream to the ICP34.5 coding region in hVG2025 are perfect match to the miRs, binding of those domains by the miR will result in degradation of mRNA (ICP34.5 in the case). Thus, we can use RT-qPCR to measure the levels of ICP34.5 mRNA to test the miR regulated ICP34.5 expression in hVG2025.

Procedure: 293 FT cell culture were transfected with miR124 and miR143 using Lipofectamine™ RNAiMAX Transfection Reagent, followed by superinfection with hVG2025. RT-qPCR was performed 24 hours post viral infection to quantify ICP27 and ICP34.5 expression and copy numbers.

Experiment Designs: 293 FT cells were transfected in triplicate with either miR124/miR143 precursors. Control cells were either transfected scrambled miR precursors (Thermo Fisher AM17110) or mock transfected.

Twenty-Four hours after transfection, cells were infected with hVG2025 at MOI=1. Cells were incubated at 37° C. 5% CO2 for 6 hours.

Cells were harvested 6 hours post virus infection for further testing. RNA was purified, and RT-qPCR was performed to measure the levels of ICP27, ICP34.5, miR124, miR143 and actin.

Results: The results of this experiment are shown in FIG. 5. While the expression levels of ICP27 was comparable in 293 FT cells that has been transfected with either miR 124/143, control/scrambled miR, or non-transfected cells, the levels of ICP34.5 were significantly lower in cells that has been transfected with miR124/143 ((p-value=0.0002).

To verify the presence of miR124 and miR143 in the transfected cells, RT-qPCR was also performed on transfected cells (infected and non-infected). High levels of miR124 and miR143 were detected the transfected cells. Moreover, it was also observed that viral infection did not affect or reduce the levels of miR in these cells:

TABLE 3
miR124/143 level during infection

| | | | CT Mean | | |
|---|---|---|---|---|---|
| | | | Actin | miR124 | miR143 |
| Virus | Condition | Duplicate | (CT Mean) | (CT Mean) | (CT Mean) |
| hVG2025, MOI = 1 | miR124/143 | 1 | 17.633 | 14.193 | 15.785 |
| | | 2 | 17.763 | 14.242 | 16.223 |
| No Virus | miR124/143 | 1 | 17.587 | 14.168 | 15.436 |
| | | 2 | 18.176 | 14.492 | 15.658 |
| | negative | 1 | 17.751 | 34.088 | 36.681 |
| | | 2 | 17.869 | 34.833 | 36.099 |

Conclusions: The results show a significant reduction in the expression of ICP34.5 in the presence of miR124/143 (p=0.0002). On the other hand, expression levels of ICP27, which is not regulated by the miRs were the same among all groups, suggesting the downregulation of ICP34.5 was miR124/143 specific. In addition, viral infection did not change the levels of miR in treated cells.

### Example 4

### Dose-Dependent, Vector-Induced Tumor Cytotoxicity in a Variety of Tumor Cell Lines

Objective: This study tested anti-tumoral activity of hVG2025, measured as cell viability and half maximal inhibitory concentration (IC50), on 11 human tumor cell lines and 6 mouse tumor cell lines by in vitro culture.

Procedure: The following cell lines were seeded into well plate at 5E3 cell/well and incubated overnight in various cell-specific suitable culture mediums as suggested by vendors:

A549 (human NSCLC), BxPC3 (human pancreatic cancer), Panc01 (human pancreatic cancer), Capan-1 (human pancreatic cancer), SW620 (human colon cancer), LnCAP and PC-3 (human prostate tumor cell), U2OS (human tibia sarcoma), HepG2 (human hepatocyte carcinoma), Kato III (human gastric cancer), SH-SY5Y (human neuroblastoma), Panc02 (mouse pancreatic cancer), Cloudman S91 (mouse melanoma), MB49-Luc (mouse bladder cancer), CT26 (mouse colon cancer), A20 (mouse reticulum sarcoma), 4T1 (mouse breast cancer).

hVG2025 were diluted and added into cell culture at MOI ranging from MOI 5, 1, 0.2, 0.04 and 0 (MOI 0 is media only as vehicle control). Incubated for 3 days. Cell viability was assayed by MTT method under the standard MTT assay

Cell viability was plotted against MOI for each cell line. IC50 of permissive cell lines were calculated by GraphPad Prism. IC50 of resistant cell lines were noted as not determinable (N.D.)

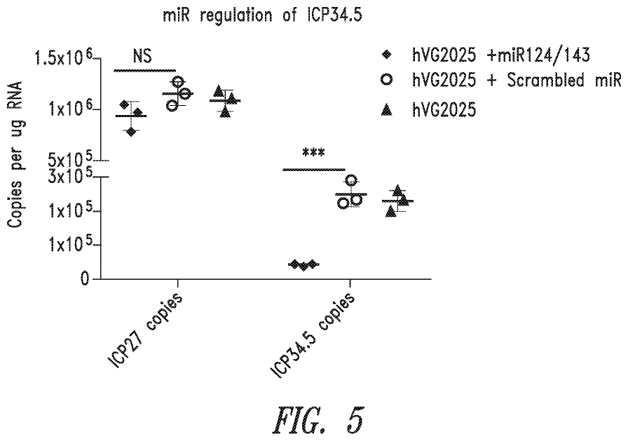

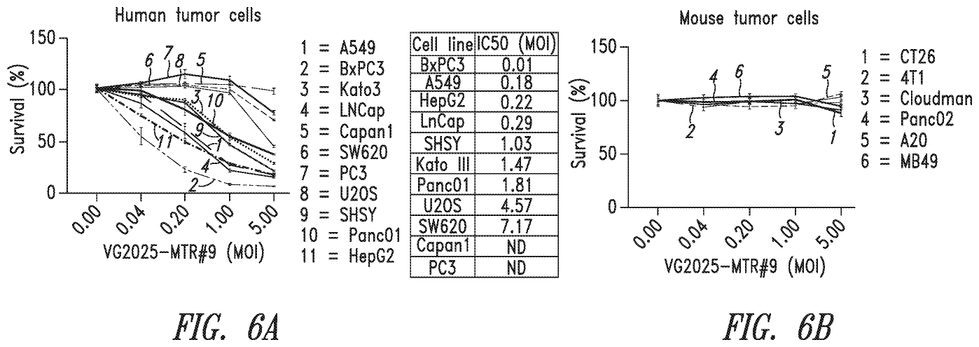

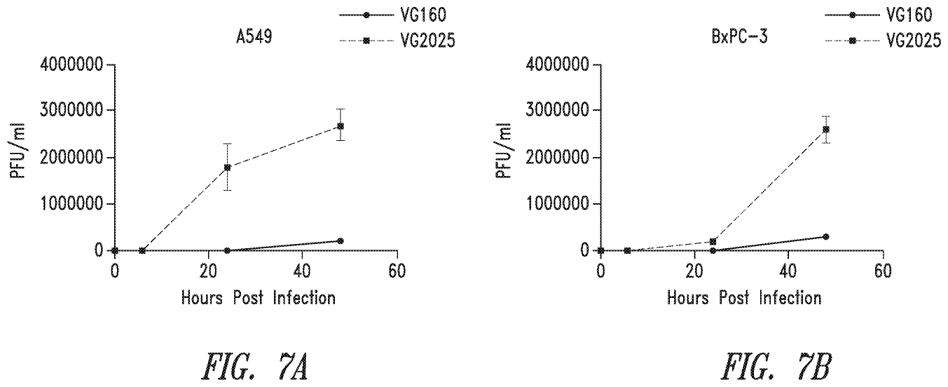

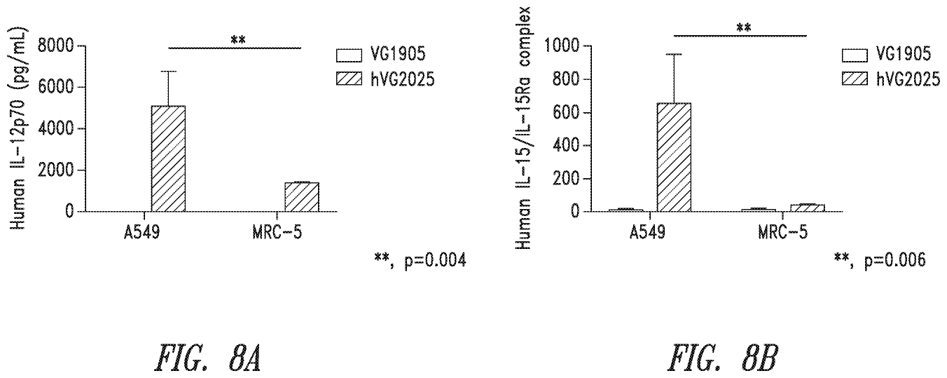

Results: The results of this experiment show the anti-tumoral activity of hVG2025, measured as cell viability on 11 human tumor cell lines (FIG. 6A) and 6 mouse tumor cell lines (FIG. 6B) by in vitro culture. More specifically, HepG2, A549, LnCap and BxPC3 were most sensitive to hVG2025 with IC50 lower than MOI 1. Capan-1, PC3 and SW620 were found to be resistant to hVG2025. Other human tumor cells were found to have intermediate permissiveness to hVG2025. All mouse tumor cells tested in this study were found to be resistant to hVG2025.

Conclusion: hVG2025 at MOI<1 is cytotoxic to several cell lines representing pancreatic (BxPC3), lung (A549), prostate (LnCaP) and hepatocellular carcinoma (HepG2). Mouse tumor cell lines are not susceptible to hVG2025 induced cytotoxicity.

### Example 5

Virus Replication Efficiency of VG2025 in Tumor Cells in Comparison with an ICP34.5-oHSV-1

Objective: This study is to compare replication efficiency of hVG2025 virus and a ICP34.5 (−) oHSV-1 strain (VG160) in A549 and BxPC3 cells.

Study design: A549 and BxPC3 were prepared with 6-well plate. After overnight incubation, hVG2025 and ICP34.5-oHSV-1 were infected the cells at MOI=0.5. Two hours after infection, medium was changed with virus free fresh medium. At 6 hours, 24 hours and 48 hours post infection, cells and medium were collected together and stored in −80° C. followed by plaque assay on Vero cells. Each time, two samples/virus/cell lines were collected.

Measurements: All the samples were prepared for plaque assay based on the SOP.

Results: Titers of hVG2025 grew in A549 and BxPC3 cells are shown in Table 4. The growth curve of the two viruses in the two tumor cell lines were shown in FIGS. 7A and 7B.

TABLE 4
Virus titer at 48h infection in 2 cell lines

| | Titer (PFU/ml) | |
|---|---|---|
| Time | A549 | BXPC3 |

| point | ICP34.5(−) | hVG2025 | ICP34.5(−) | hVG2025 |
|---|---|---|---|---|
| 0 | 478 | 890 | 1 | 1 |
| 6 | 2020 | 4960 | 1 | 1 |
| 24 | 20200 | 1790000 | 54000 | 207000 |
| 48 | 220000 | 2690000 | 310000 | 2600000 |

Conclusion: The results showed that the replication of hVG2025 was about 10-fold higher than VG160 (ICP34.5−) in A549 and BXPC3 cells (Both are CEA high expressors at 48 hours post infection). Since the main differences of the two viruses in relevance to this assay are a) The virus essential gene ICP27 is controlled by a CEA promoter for hVG2025 but the same gene is controlled by its native viral promoter for VG160; b) the ICP34.5 gene is regulated by miR124 and miR143 in hVG2025 but is deleted in VG160. Therefore, the replication advantage shown by hVG2025 in the two tumor cell lines may be attributed to both increased ICP27 transcription and a functioning ICP34.5.

### Example 6

Human IL-12p70 and Human IL-15/IL-15Ra Expression from HVG2025 Virus Infected Tumor Cells

Objective: To measure human IL-12p70 and human IL-15/IL-15Ra payload secretion from hVG2025 virus infected tumor cell lines.

Procedure: A549 (NSCLC) and MRC-5 (fibroblast) cell lines were seeded in 12-well plate at 37° C. for overnight and subsequently infected with hVG2025 virus at MOI=1 for 24 hours. Cell lines infected with VG1905 backbone virus (no human IL-12p70 and human IL-15/IL-15Ra) at the same condition was used as negative control. Twenty four-hour post-virus infection, supernatants were harvested from cells and human IL-12p70 and human IL-15/IL-15Ra secretion were quantified by ELISA assays.

Results: After 24 hours of hVG2025 virus infection, payload expression was observed in A549 and MRC-5 cells. However, A549 cells produced significantly higher human IL-12p70 and human IL-15/IL-15Ra payloads than MRC-5 cells (3.6-fold human IL-12p70 and 14.6-fold human IL-15/IL-15Ra, respectively). (FIGS. 8A and 8B and Table 5).

TABLE 5
Raw data of ELISA assays

| | | 76.A549 | | 77.MRC5 | |
|---|---|---|---|---|---|
| 75. | | 78.Average | 79.SD | 80.Average | 81.SD |
| 82.L-12p70 | 83.VG1905 | 84.1.2 | 85.2.39 | 86.0 | 87.0 |
| | 88.hVG2025 | 89.5085.8 | 90.1690.17 | 91.1395.3 | 92.35.83 |
| 93.L-15/ | 94.VG1905 | 95.14.4 | 96.2.95 | 97.16.7 | 98.2.11 |
| IL15Ra | 99.hVG2025 | 100.655.2 | 101.294.88 | 102.44.9 | 103.3.35 |

Conclusion: A549 cells infected with hVG2025 virus produce more human IL-12p70 and IL-15/IL-15Ra payloads compared to infected MRC5 cells.

### Example 7

Biological Function of hVG2025 Payloads: Human IL-12p70 and Human IL-15/IL-15Ra

Objective: To test biological function of human IL-12p70 with human IL-15/IL-15Ra payloads produced from hVG2025 virus infected cells.

Procedure: Human IL-12p70 and human IL-15/IL-15Ra payload-expressing hVG2025 or VG1905 backbone (no human IL-12p70 and human IL-15/IL-15Ra) viruses were used to infect Vero cells at MOI of 1. Supernatant of infected Vero cell cultures was collected at 48 hours post-infection and payloads expression was measured by ELISA assays before being used for the following procedure. Human PBMCs were pre-stimulated with 0.25 mg/ml of mitogen phytohemagglutinin (PHA) at 37° C. incubator for 24 hours. Next day, PHA-stimulated human lymphoblasts were co-incubated with different volumes of virus-infected Vero supernatant for 48 hours. Supernatants from co-incubation were harvested and human IFN-g secreted from immune cells was quantified by human IFN-g ELISA assay.

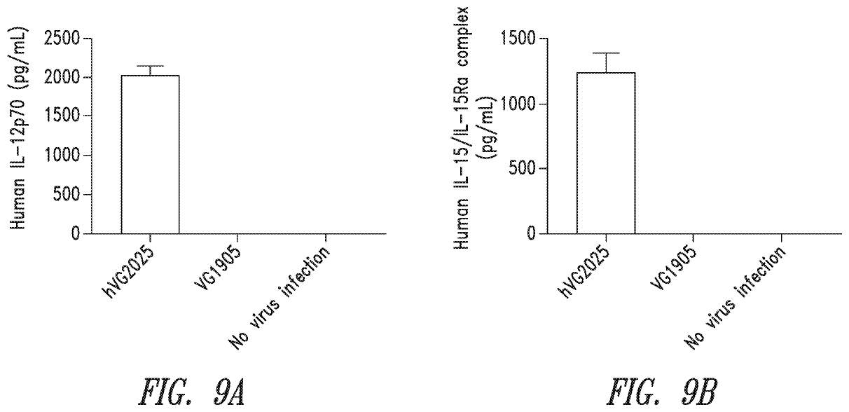

Results: Payload expression in virus infected Vero supernatant was determined by ELISA prior to examining payload bioactivity. Only supernatant harvested from hVG2025 infected cells produced human IL-12 and IL-15/IL-15Ra but not in VG1905 infected or no virus infected supernatant (FIGS. 9A and 9B and Table 6).

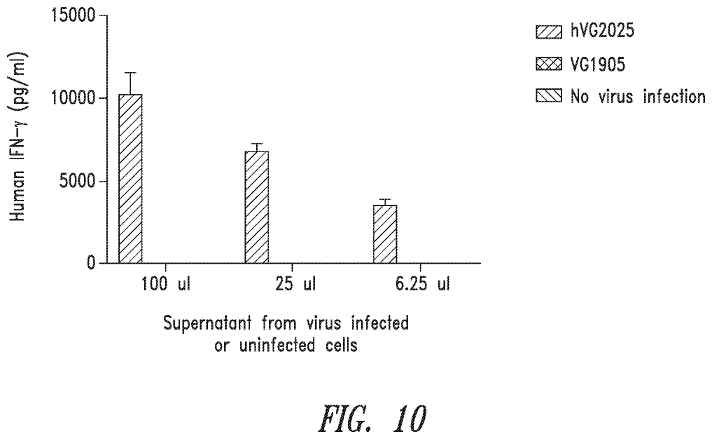

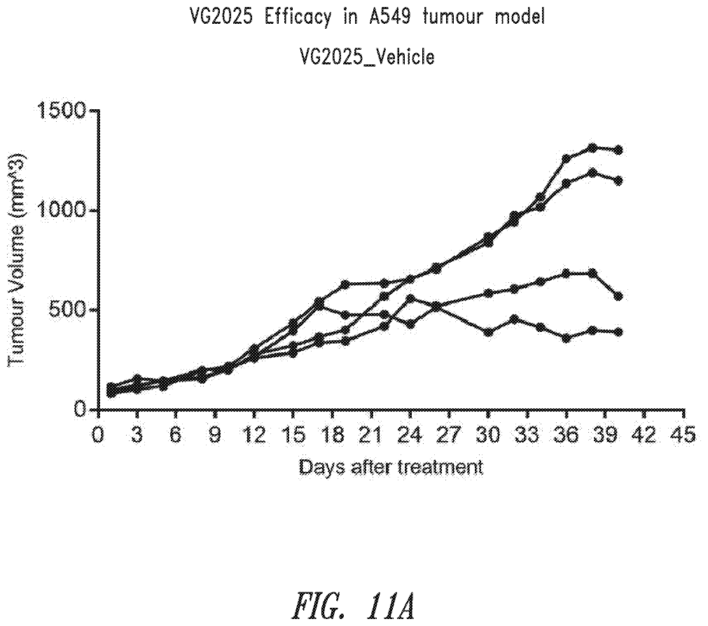

Next, we tested payload bioactivity based on human IFN-g secretion by PBMCs. Result showed a dose-dependent human IFN-g production from PHA-stimulated lymphoblasts co-incubated with supernatant harvested from hVG2025 virus infected cells whereas no IFN-g secretion was detected from PHA-stimulated lymphoblasts exposed to supernatants from cells infected with either hVG2025, VG1905 or no virus (FIG. 10 and Table 7).

TABLE 6

| | Human IL-12p70 | | Human IL-15/IL-15Ra | |
|---|---|---|---|---|
| | AVG | SD | AVG | SD |
| hVG2025 | 2031.8 | 128.3 | 1242.0 | 149.9 |
| VG1905 | 0 | 0 | 0.0 | 0.0 |
| No virus infection | 0 | 0 | 8.5 | 12.1 |

TABLE 6
Raw data of human IFN-g production from PHA-stimulated lymphoblasts

| | | Supernatant harvested from | | | | | |
|---|---|---|---|---|---|---|---|
| | | hVG2025 | | VG1905-TK #1 | | No infection | |
| | | AVG | SD | AVG | SD | AVG | SD |
| Volume of supernatant for | 100 ml | 10224 | 1832.8 | 0 | 0 | 0 | 0 |
| co-incubation with PHA- | 25 ml | 6748 | 676.0 | 0 | 0 | 0 | 0 |
| stimulated lymphoblasts | 6.25 ml | 3477.5 | 597.5 | 0 | 0 | 0 | 0 |

Conclusion: Human IL-12p70 and human IL-15/IL-15Ra payloads were secreted from hVG2025 virus infected Vero cells. The supernatant containing above secreted payloads from hVG2025 infected cells stimulate human PBMCs to produce IFN-g.

### Example 8

TCID50s of hVG2025 in A549 and MRC5 cells

Objectives: To determine virus titers of hVG2025 in A549 and MRC5 cells by TCID50 assay, and to find infection and replication difference of hVG2025 in tumor and normal cell lines.

Study design: 6 times of repeat dilution of virus as following: add 10 μl of virus hVG2025 from −80° C. stocking in 990 μl of DMEM medium to produce 100 times of dilution for first tube, following 7 tubes in 10 times of series dilution by adding 100 μl of virus in with 900 μl of DMEM medium, total 48 tubes of virus dilution preparation. Repeat the same dilution of virus by two people. Add 100 μl/well of diluted virus to infect A549 or MRC5 cells in 96 well plate, total 96 wells of each cell line were infected. Each dilution group infected 12 repeated wells. After 3 to 5 days of inoculation at 37° C. 5% CO2, visualize and calculate TCID₅₀ under microscope.

Measurements: Cytopathic effect (CPE) was visualized under an inverted microscope by observing wells inoculated with virus dilutions. The TCID50 was calculated based on the method of Reed and Muench.

TABLE 7
TCID50 of VG2025 on A549 and MRC5

| Cell line | TCID50 | MRC5 comparing to A549 |
|---|---|---|
| A549 | 7.00E−08 | N/A |
| MRC5 | 5.35E−06 | MRC5 is 76.43-fold higher |

Conclusions: TCID50 of hVG2025 in nontumorous MRC5 cells is much higher than in A549 cell with a difference of 76.43-fold, indicating a significantly higher replication efficiency of VG2025 in A549 tumor cells.

### Example 9

Treatment of A549 Burdened BALB/c Nude Mice with hVG2025

Objective: This study is to determine the dose dependent anti-tumor efficacy and survival benefits of intra-tumoral delivered hVG2025 virus in a A549 lung cancer xenograft model in athymic mice.

Study design: 29 SPF-grade female Balb/c-nude mice were subcutaneously injected with 5×10\hat{ }6 A549 cells/mouse and randomized into 6 groups, 5 mice in each virus treatment group (4 mice in Vehicle Group). Group 1 was vehicle (PBS) control. Group 2˜5 were test groups, administered with a single dose of hVG2025 at dose of 10\hat{ }2, 10\hat{ }3, 10\hat{ }4, 10\hat{ }5, 10\hat{ }6 PFU/mouse, respectively via intratumor injection. All animals were appropriately identified by marking on different body parts, housed, fed according to standard protocols.

Measurements: All mice were observed at least twice daily after administration for clinical findings. Body weight and tumor size were measured at baseline and then 2˜3 times per week. Data were expressed as Means±SEM.

Animals were sacrificed if tumor grew to 1500 mm3 (measured by caliper) or showed Grade 5 clinical symptoms.

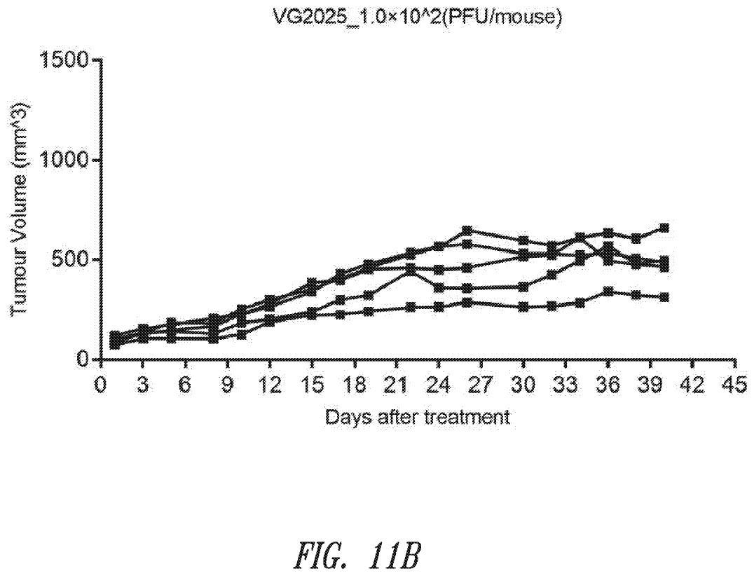

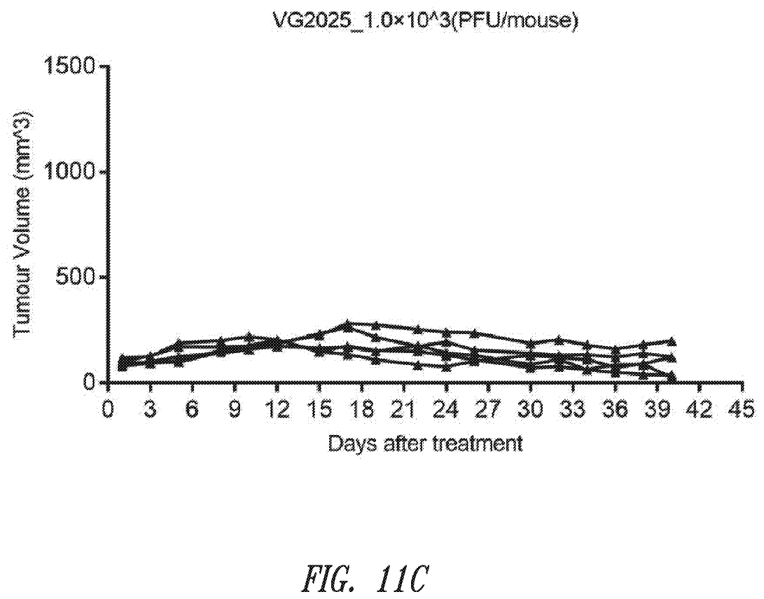

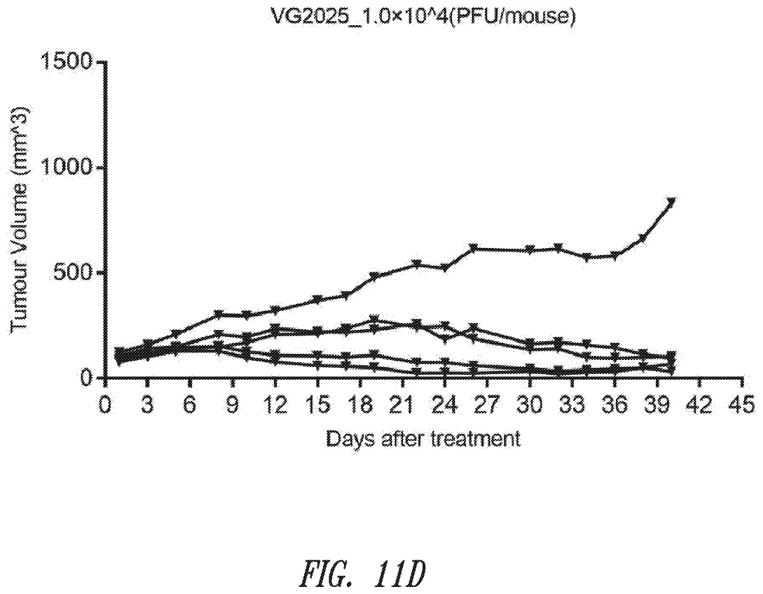

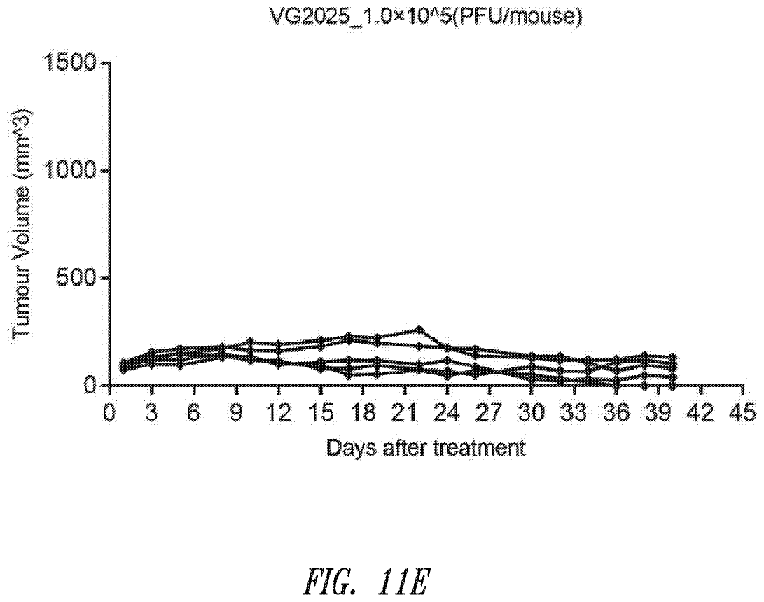

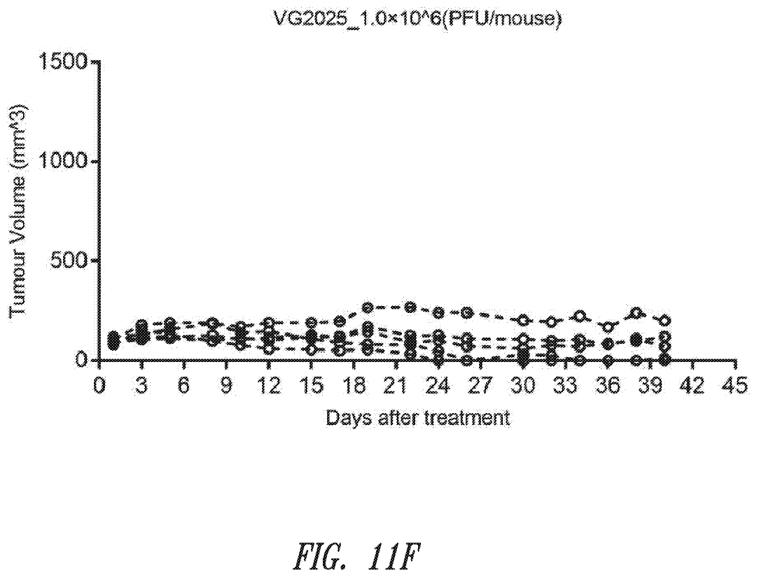

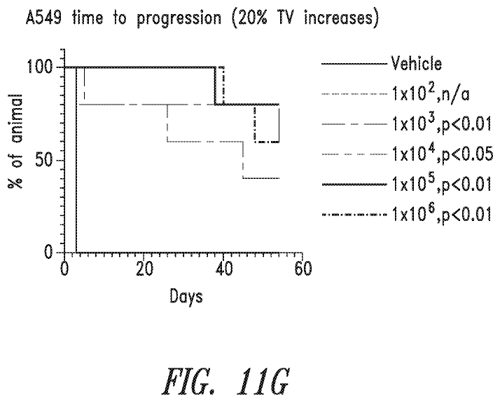

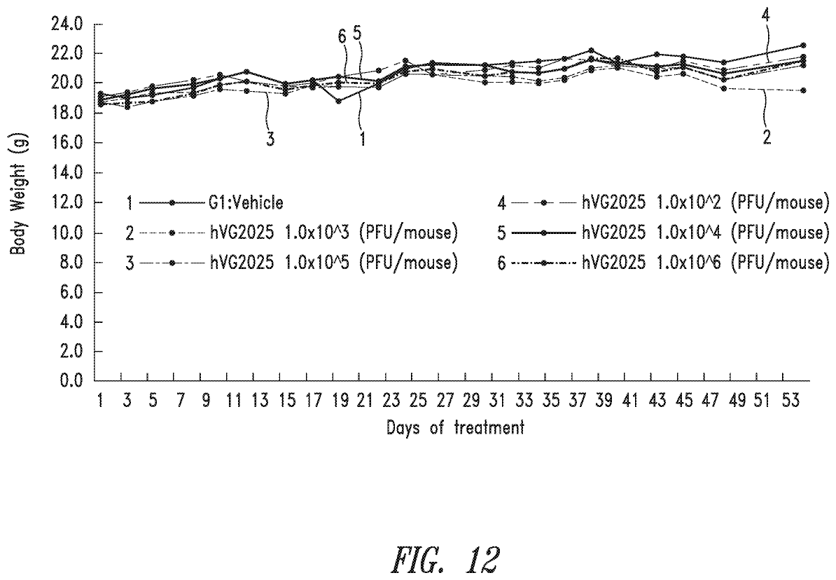

Results: Comparing to vehicle control group, tumour growth inhibition was observed following intra-tumoral treatment with 10\hat{ }3, 10\hat{ }4, 10\hat{ }5, 10\hat{ }6 PFU/mouse of hVG2025 (see FIGS. 11A to 11G). Tumor sizes were measured up to day 40 after implantation and terminated due to spontaneous tumor regression in some vehicle control animals. The mice survived to day 54 and sacrificed as scheduled. No statistical analysis was performed on tumor sizes as the sample sizes were too small to determine whether it is normally distributed. Instead, duration of tumor inhibition in each group was analyzed with Gehan-Breslow-Wilcoxon test. Table 8 provides a summary of tumor regression in each group, and FIG. 12 graphically depicts changes in body weight after hVG2025 treatment.

TABLE 8
Summary of tumor regression mice in each group

| | Number of mice with | Overall response |
|---|---|---|
| Group | complete tumor regression | and tumor control |
| Vehicle | 0/4 | 0/4 |
| 10\hat{ }2 PFU/mouse | 0/5 | 1/5 |
| 10\hat{ }3 PFU/mouse | 0/5 | 5/5 |
| 10\hat{ }4 PFU/mouse | 0/5 | 4/5 |
| 10\hat{ }5 PFU/mouse | 1/5 | 5/5 |
| 10\hat{ }6 PFU/mouse | 2/5 | 5/5 |

Conclusion: Anti-tumor activity of hVG2025 was shown in mouse model xenografted with human lung cancer with a dose-dependent fashion. It appeared that virus dose higher than 10\hat{ }3 PFU/mouse is sufficient to show the inhibition effect. But higher dose had more mice with complete tumor regression. No toxic symptoms were seen in mice of any group.

### Example 10

Treatment of BxPC3 burdened BALB/c nude mice with hVG2025

Objective: This study is to determine the anti-tumor efficacy and survival benefits of intra-tumoral injectable hVG2025 virus in a BxPC3 pancreatic cancer xenograft model of athymic mice.

Study design: 30 SPF-grade female Balb/c-nude mice were subcutaneously injected with 5×10\hat{ }6 BxPC3 cell/mouse and randomized into 6 groups, 5 mice in each group. Group 1 was vehicle control, intratumorally injected with PBS. Group 2˜5 were test groups, administered with a single dose of hVG2025 at 10\hat{ }2, 10\hat{ }3, 10\hat{ }4, 10\hat{ }5, 10\hat{ }6 PFU/mouse, respectively via intratumoral injection. All animals were appropriately identified by marking on different body parts, housed, fed according to standard protocols.

Measurements: All mice were observed at least twice daily after administration for clinical symptoms. Body weight and tumor size were measured at baseline and then 2˜3 times per week. Data were expressed as individual tumour size.

Animals were sacrificed if tumor grew to 1500 mm3 (measured by caliper) or showed Grade 5 clinical symptoms.

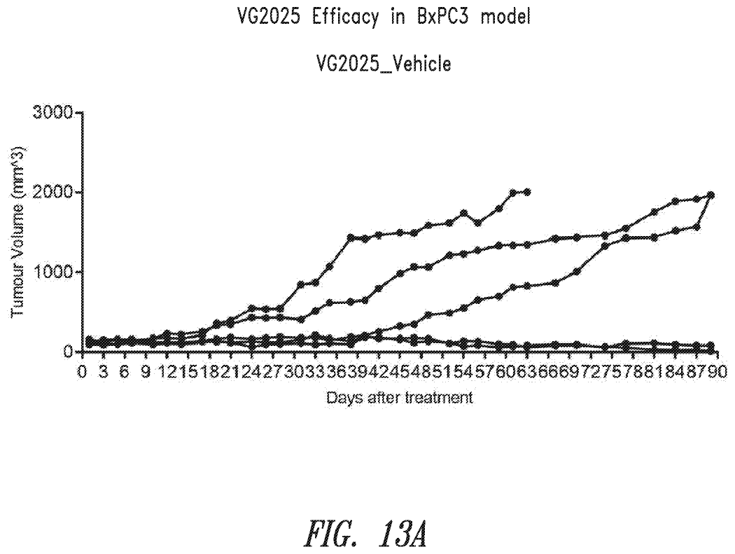

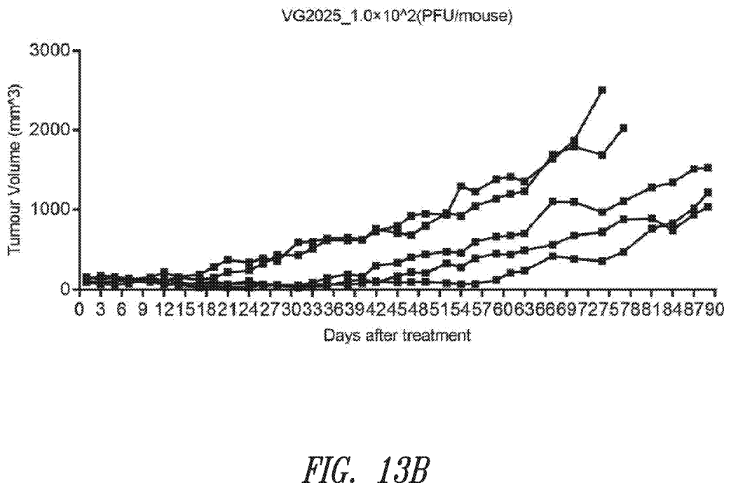

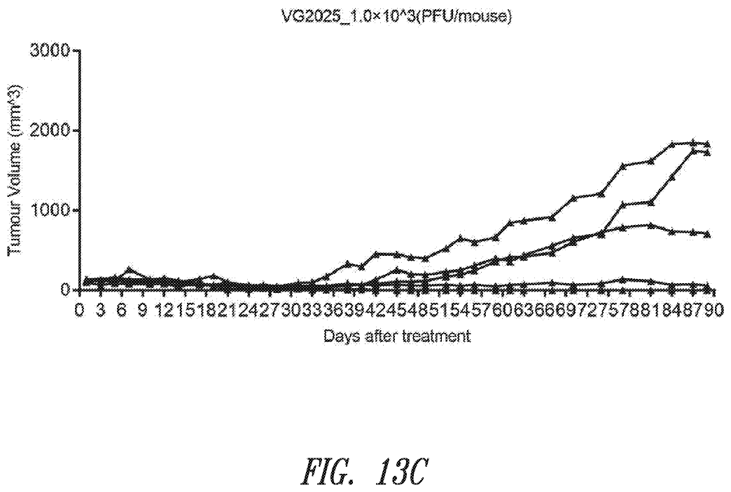

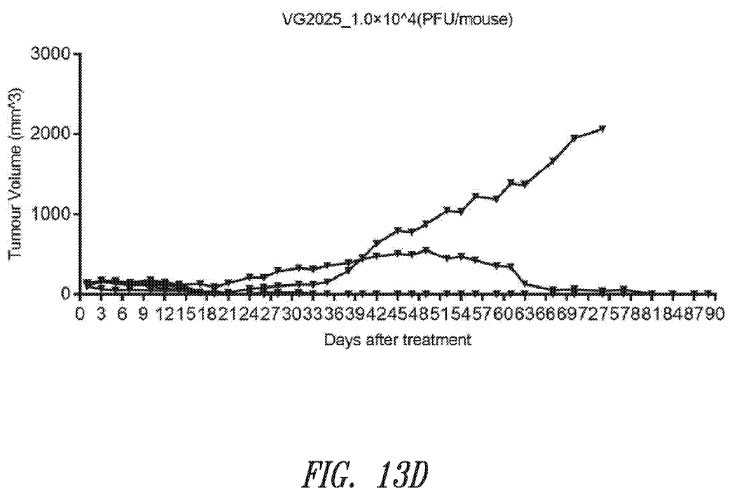

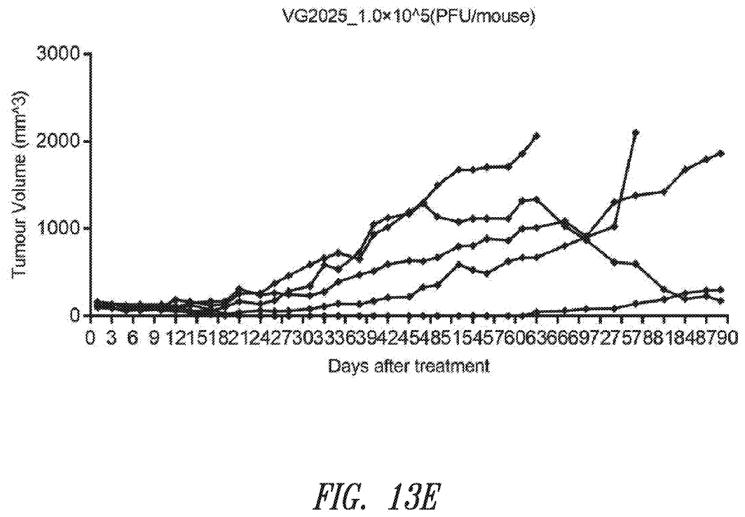

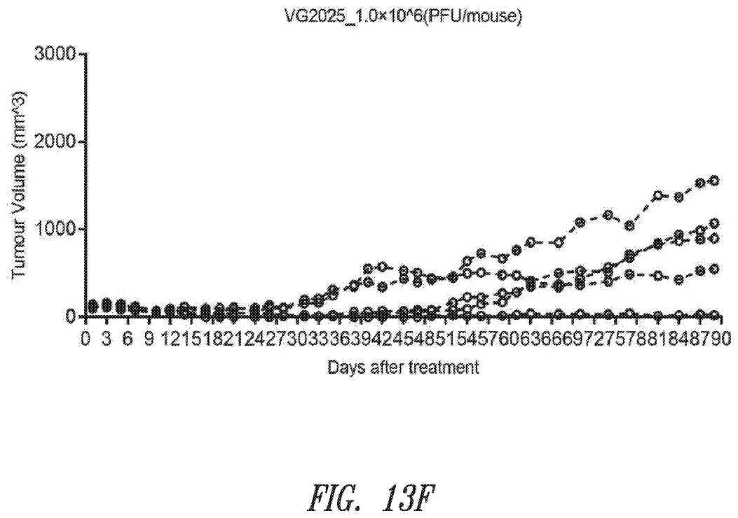

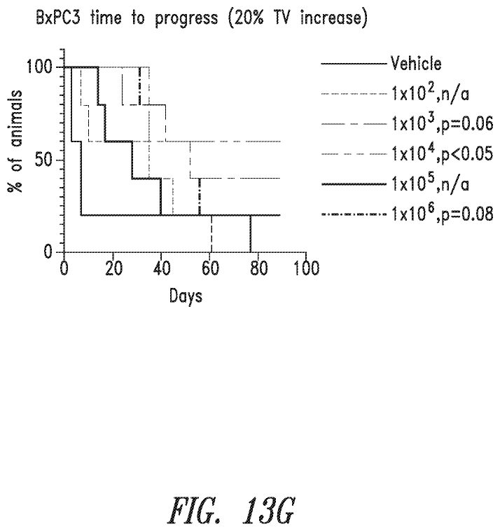

Results: are provided in FIGS. 13A to 13G. Comparing to vehicle control group, BxPC3 tumour growth inhibition was observed following intra-tumoral treatment with 10\hat{ }3, 10\hat{ }4, 10\hat{ }5, 10\hat{ }6 PFU/mouse of hVG2025. Mice were euthanized only due to tumour burden and the rest were allowed to survive to day 89 post implantation. Tumor failed to grow in 2/5 animals in the vehicle control group.

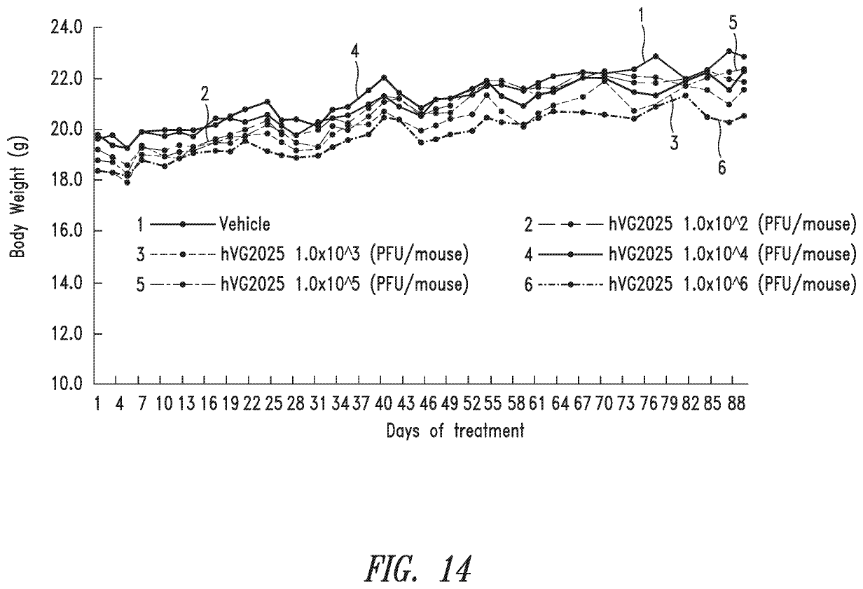

No statistical analysis was performed on tumor sizes as the sample sizes were too small to determine whether they were normally distributed. Instead, duration of tumor inhibition in each group was analyzed with Gehan-Breslow-Wilcoxon test (FIG. 14). There was no significant difference in body weight of mice among all groups. No clinical symptoms were observed.

Conclusions: Anti-tumor activity of hVG2025 was apparent in nude mouse xenografted with human pancreatic cancer. Some animals received hVG2025 showed complete tumor regression. Larger sample size is needed to show significant efficacy due to large variation among tumor growth in the animals. hVG2025 was safe in tumor bearing nude mice.

### Example 11

Comparison of Antitumor Efficacy of hVG2025 and ICP34.5 Deleted HSV-1 in Lung Cancer (A549) Model

Objective: The experimental goal is to compare the anti-tumor efficacy of hVG2025 and an ICP34.5 (−) oHSV-1 in athymic A549 xenograft mouse model.

Study design: 30 SPF-grade female athymic nude mice were subcutaneously injected with 2.5×10⁶ A549 cells per mouse and randomized into 5 groups, with 6 mice in each group. Group 1 was vehicle control, intratumorally injected with vehicle containing 1×DPBS+7.5% glycerin. Group 2 to 5 were test groups; groups 2 to 4 were administered with a single dose of hVG2025 at 5×10³, 5×10⁵ and 5×10⁷ PFU/mouse, respectively. Lastly, group 5 was administered with single dose of an ICP34.5 gene deleted HSV-1 vector at 5×10⁷ PFU/mouse. All test-group administrations were via intratumoral injections. All animals were appropriately housed and fed according to standard protocols and were appropriately identified by markings on different body parts.

Measurements: All mice were observed at least twice daily after administration for clinical findings. Body weight and tumor size were measured at baseline and then 2˜3 times per week. Statistical analysis was performed with GraphPad Prism 7.03. Tumor volume was measured using a vernier caliper (Length×Width×Depth×0.5236). Data shown are tumor volume (mm3) and values are mean±SEM. Tumor regression statistical significance (P<0.05) was determined using a Multiple t-test. Animals were sacrificed if tumor grew to 1000 mm3 or showed Grade 5 clinical symptoms.

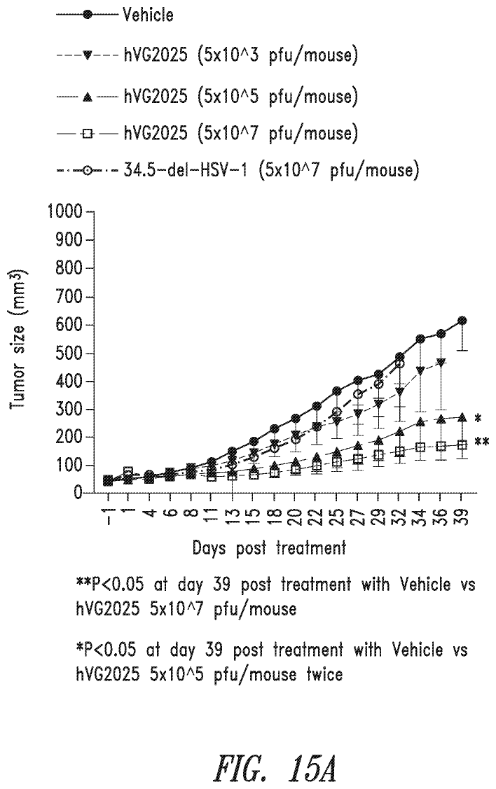

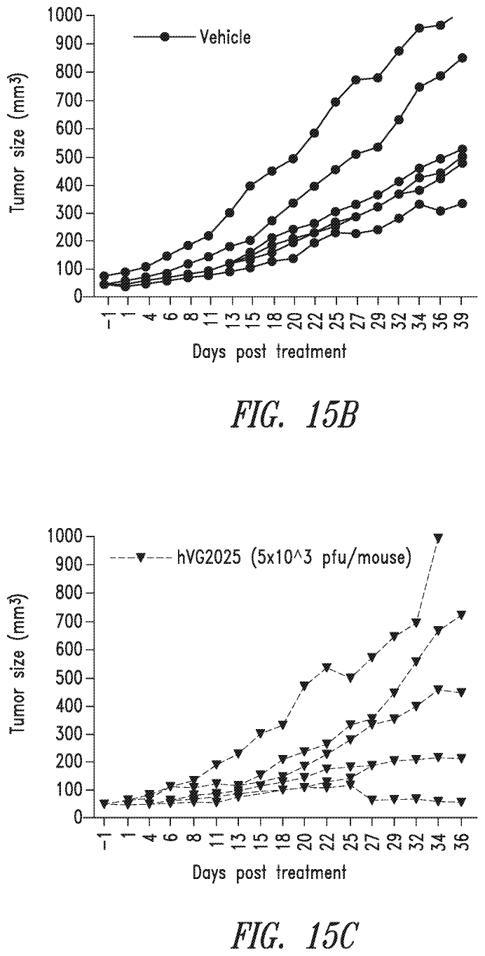

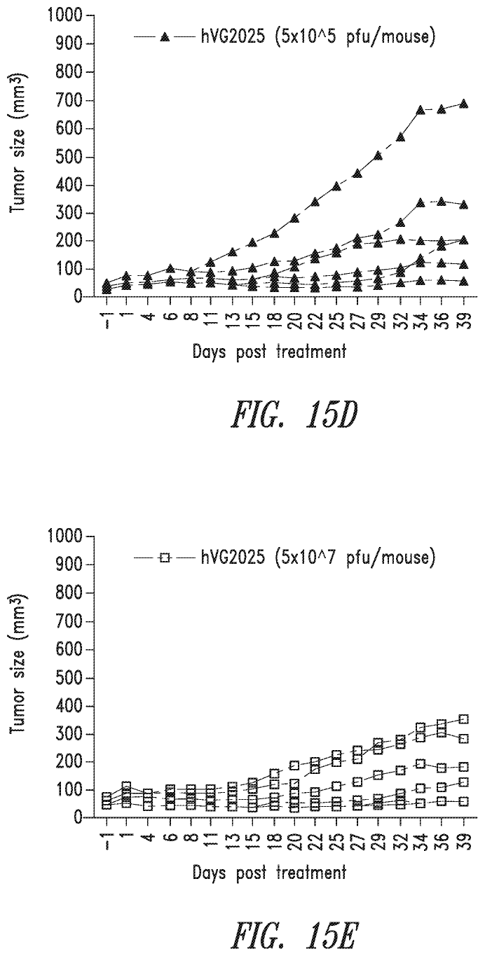

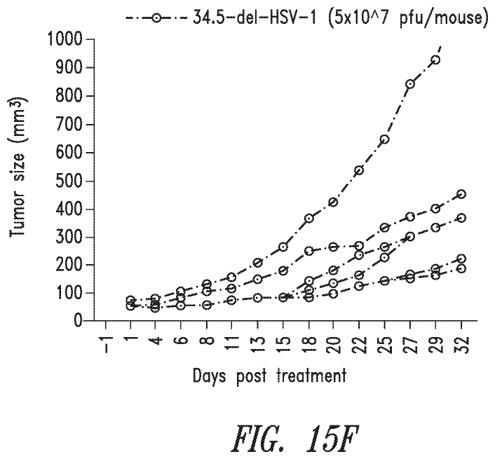

Results: The results are provided in FIGS. 15A, 15B, 15C, 15D, 15E and 15F. Briefly, compared to the vehicle control group, statistically significant tumor growth inhibition at 39 days post-treatment was observed following single intra-tumoral treatments with 5×10⁵ and 5×10⁷ PFU/mouse. The results showed that, in comparison to tumor inhibition effect of ICP34.5-deletion mutant at the dose of 5×10⁷ PFU/mouse, hVG2025 demonstrated much better efficacy even at the dose 100-fold lower than the ICP34.5-mutant.

Conclusion: Anti-tumor effect of hVG2025 was confirmed in mouse model xenografted with human lung cancer with a dose-dependent manner. Tumor growth was significantly controlled at dose of 5×10⁵ and 5×10⁷ PFU/mouse. Significantly augmented antitumor effect is observed by hVG2025 compared to the ICP34.5 gene deleted HSV-1 vector.

### Example 12

Survival of Young DBA/2 Mice after Subcutaneous Inoculation with VG2025

Objective: This study is to determine the toxicity of hVG2025 after subcutaneous inoculation in young DBA/2 mice.

Study design: 4 weeks-old young 30 SPF-grade female DBA/2 mice were randomized into 6 groups, 5 mice in each group. Group 1 was vehicle control, subcutaneously injected with PBS. Group 2˜4 were test groups, administered with a single dose or multiple doses administrated for 5 constitutive days (Group 4 only) of the test article at different doses via subcutaneous injection as indicated in Table 9. Group 5˜6 were positive groups, administered with a single dose wild-type 17+ HSV virus.

TABLE 9
Grouping for subcutaneous injection of hVG2025 on DBA/2 mice

| Group | Test article | Dose (PFU/mouse) | Frequency |
|---|---|---|---|
| 1 | Vehicle | 0 | D1 |
| 2 | hVG2025 | 10\hat{ }6 | D1 |
| 3 | hVG2025 | 10\hat{ }8 | D1 |
| 4 | hVG2025 | 10\hat{ }8 | D1~D5 |
| 5 | Wild-type 17+ | 10\hat{ }5 | D1 |
| 6 | Wild type 17+ | 10\hat{ }6 | D1 |

All animals were appropriately identified by marking on different body parts, housed, fed according to standard protocols.

Measurements: All mice were observed at least twice daily after administration for clinical findings. Body weight was measured at baseline and then 2˜3 times per week. Data were expressed as mean bodyweight. Survival curve was showed in each group.

Animals were sacrificed if bodyweight reduced by 20% or showed Grade 5 clinical symptoms.

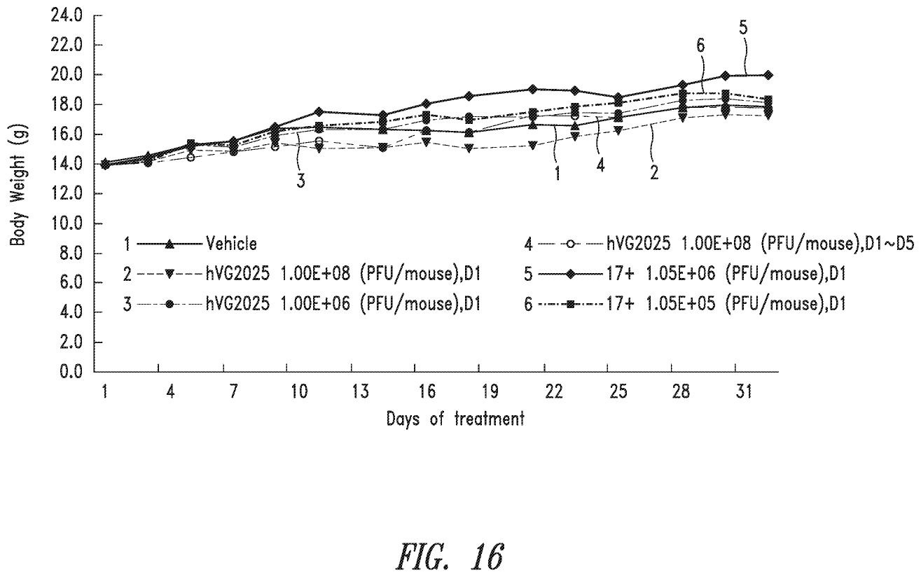

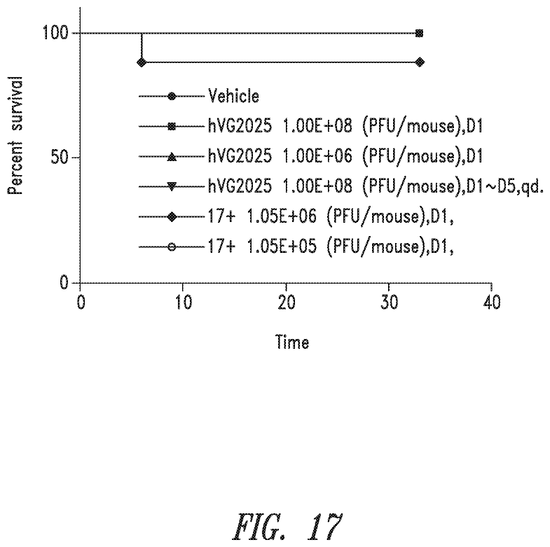

Results: During the entire experiment, no abnormality in general behavioral activities was observed. Comparing to vehicle control group, there was no significant difference in body weight of mice among groups as shown in FIG. 16. only one mouse in the 17+10\hat{ }6 PFU/mouse treatment group found bodyweight loss and morbidity 5 days after inoculation, the mouse was found dead despite special care was given. The percent survival is shown in FIG. 17.

Conclusion: Some toxicity was seen in 17+ strain at 10\hat{ }6 PFU/mouse resulted in one animal death. No toxicity was observed in any animals injected with hVG2025 subcutaneously even at 100-fold titers higher than the wild-type and with 10\hat{ }8 PFU injected once a day for constitutive 5 days (group 4). Therefore, hVG2025 is safe in young DBA/2 mice administrated through subcutaneous route.

### Example 13

### Neurovirulence Assay of VG2025 by Nasal Inoculation in Young DBA/2 Mice

Objective: This study is to determine the toxicity of hVG2025 comparing to wild-type parental strain 17+ and VG161, an ICP34.5 (−) oncolytic HSV-1, through nasal inoculation in young DBA/2 mice. Since nasal site is innervated by both trigeminal ganglion and olfactory bulb, this model is very sensitive to test HSV neurovirulence.

Study design: 4-weeks-old young 30 SPF-grade female DBA/2 mice were randomized into 6 groups, 5 mice in each group. Group 1 was vehicle control, nasal inoculation with PBS. Group 2 was a positive control, inoculated with lethal dose of wide type ICP34.5 positive 17+ strain. Group 4-6 were test groups administered with a single dose of 2 levels of hVG2025 or VG161 via nasal inoculation. (Table 10).

TABLE 10
Grouping of nasal inoculation of DBA/2 mice

| Test article | Dose (PFU/mouse) | Frequency |
|---|---|---|
| Vehicle | 0 | D1 |
| 17+ | 10\hat{ }5 | D1 |
| VG161(ICP34.5−) | 10\hat{ }5 | D1 |
| hVG2025 | 10\hat{ }5 | D1 |
| VG161(ICP34.5−) | 10\hat{ }7 | D1 |
| hVG2025 | 10\hat{ }7 | D1 |

All animals were appropriately identified by marking on different body parts, housed, fed according to standard protocols.

Measurements: All mice were observed at least twice daily after administration for clinical findings. Body weight was measured at baseline and then 2˜3 times per week. Data were expressed as mean bodyweight. Survival curve was showed in each group.

Animals were sacrificed if bodyweight reduced by 20% or showed Grade 5 clinical symptoms.

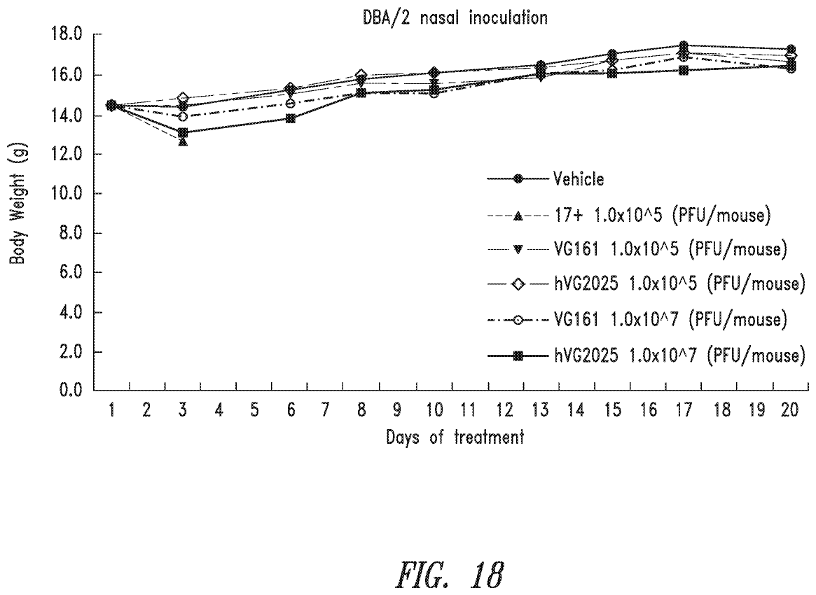

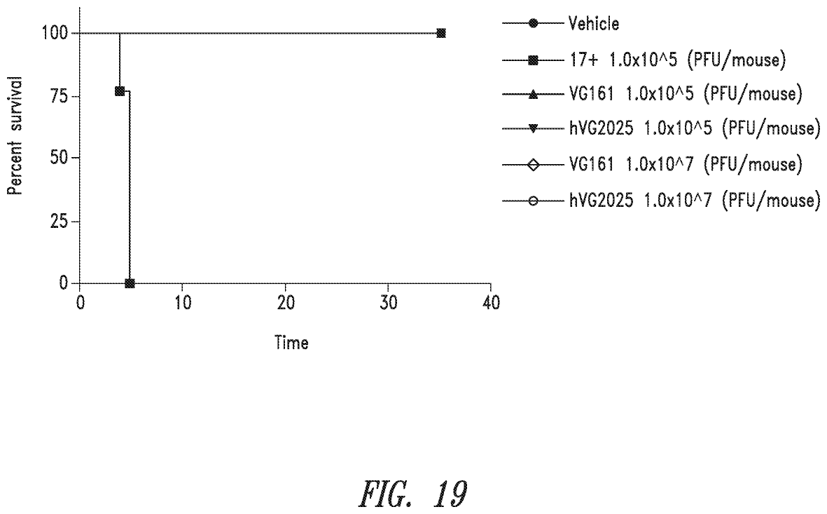

Results: During the entire experiment, no abnormality in general behavioral activities was observed in hVG2025 and VG161 treatment groups. Comparing to vehicle control group, there was no significant difference in body weight of mice among the VG161 and hVG2025 groups and all mice in both dose levels survived, as shown in FIG. 18. All mice in the 17+ treatment group were shown morbidity and bodyweight loss 3 days after inoculation and had to be sacrificed on day 6. The survival curve was shown in FIG. 19.

Conclusion: 17+ strain at 10\hat{ }5 PFU/mouse showed toxicity 3 days after the inoculation for all mice and had to be killed on day 6. hVG2025 and ICP34.5 (−) VG161 groups, including 10\hat{ }7 PFU/mouse treatment group, did not show any toxicity. Both hVG2025 and VG161 showed good safety in young DBA/2 mice, regardless of the difference in ICP34.5 status.

The safety of the miR124/143 regulated ICP34.5 expression by hVG2025 in the nervous system is demonstrated.

### Example 14

### Survival and Viral Gene Expression in Trigeminal Ganglia of BALB/c Mice Exposed to VG2025 Via Corneal Scarification to Evaluate Neurovirulence

Objectives: This study is to evaluate neurovirulence of hVG2025 using a cornea scarification model in normal BALB/c mice

Study Design: The viruses tested were a) hVG161, a HSV-1 with both copies of ICP34.5 gene deleted; b) hVG2025, a HSV-1 with CEA promoter driving ICP27 and miR124/143 regulating ICP34.5. Both virus strains also express IL12/IL15; c) HSV-1 17+ wild-type, the parental virus of hVG161 and hVG2025. A total of 140 6-week-old BALB/c mice were randomly divided into 8 groups: 1) Mock (n=10), 2) 17+ (10\hat{ }5 pfu/eye, n=30), 3) hVG161 10\hat{ }5 pfu/eye (n=30), 4) hVG161 10\hat{ }6 pfu/eye (n=10), 5) hVG161 10\hat{ }7 pfu/eye (n=10), 6) hVG2025 10\hat{ }5 pfu/eye (n=30), 7) hVG2025 10\hat{ }6 pfu/eye (n=10), 8) hVG2025 10\hat{ }7 pfu/eye (n=10). All animals received 5 μl of PBS or virus solutions at indicated doses through cornea scarification. 3 animals from group 1), 2) 3) and 6) were killed on day 5 post infection and the trigeminal ganglia (TG) and the olfactory bulbs (OB) were collected for RNAseq analysis. The rest of animals were allowed to survive to day 28 before sacrifice.

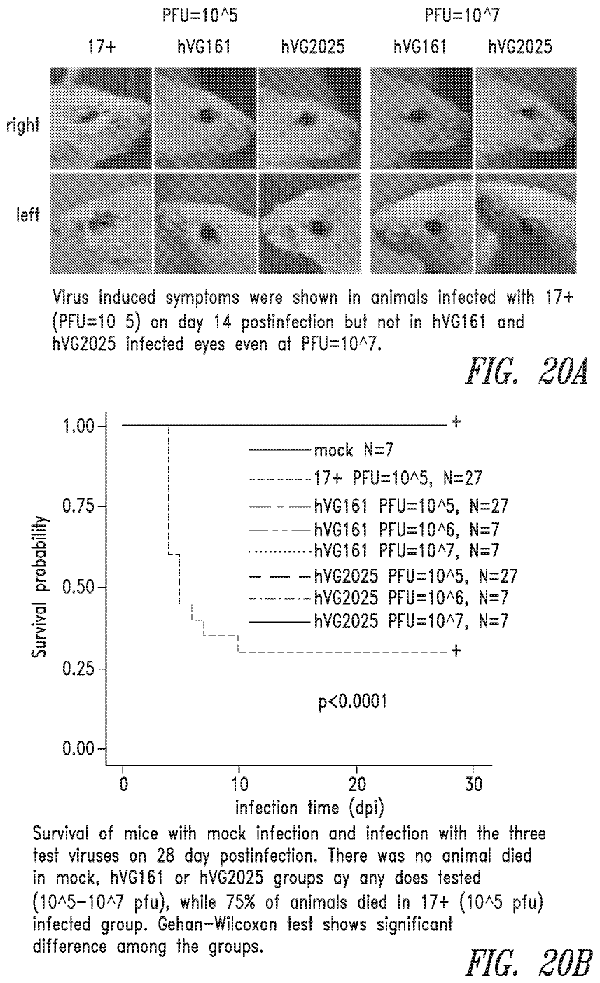

Results: Similar to mock infected animals, both hVG161 and hVG2025 inoculated mice showed no observable symptoms, while the wild type 17+ exhibit severe cornea lesions (representative picture in FIG. 20A) and ˜75% lethality within the 28 day post-infection (FIG. 20B).

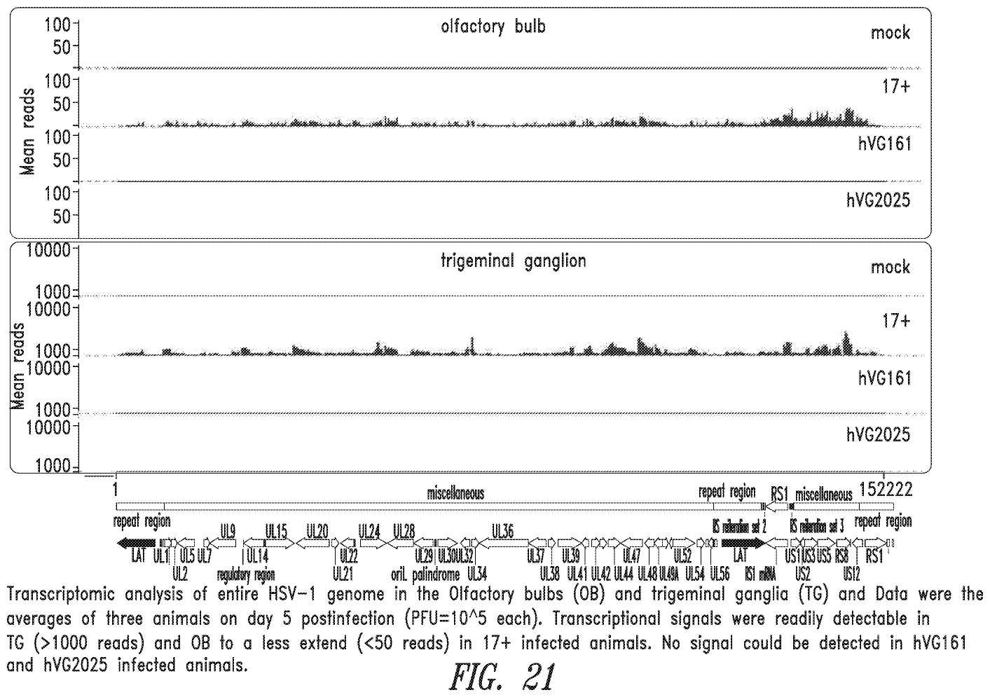

No HSV-1 transcripts from either hVG161 or hVG2025 infected mice could be detected in trigeminal ganglia and Olfactory bulbs at day 5 post-infection while the 17+ strain express high levels of essentially all HSV-1 transcripts in the trigeminal ganglia and to less extent, in the olfactory bulbs (FIG. 21).

Conclusions: 1. The cornea scarification model was effective for testing HSV-1 induced neurovirulence as a wild-type strain 17+ caused severe cornea inflammation, tissue damage and high mortality at 10\hat{ }5 pfu virus infection per mouse.

2. The engineered HSV-1 strains, either by ICP34.5 deletion (hVG161) or by transcriptional and translational dual regulation (hVG2025) did not show any virulence in both the eyes and CNS even at a 100-fold higher dose than the wild-type.

3. Expression of all viral genes was readily detectable in the trigeminal ganglia in animals corneally infected with the wild-type HSV-1 17+ infected at 10\hat{ }5 pfu on day 5 post-infection. Low levels of transcriptional activity (˜200-fold less than TG) can also be detected in the olfactory bulbs at this early infection stage, suggesting rapid and widespread dissemination of the virus in the brain.

4. Neither hVG161 nor hVG2025 infection in the cornea resulted in any detectable viral gene expression in the trigeminal ganglia or olfactory bulbs, indicating that neither of the viruses replicated in the CNS in the acute infection phase.

5. hVG2025 with regulated ICP34.5 expression is at least as safe as ICP34.5 (−) hVG161 in the nervous system.

### Example 15

### Sensitivity to Ganciclovir

Objective: The objective of this study is to test hVG2025 #9 sensitivity to ganciclovir.

Study design: Stocks of hVG2025 (tk+) and its parental strain VG1925 #2-1 (tk−) were used at 2000, 400 and 80 pfu/ml to infect Vero cells in the presence of different concentrations of ganciclovir (GCV). Number of plaques in the plates were counted as the measurement of the sensitivity to GCV.

Measurements: hVG2025 or VG1925 #2-1 viruses were diluted to designated virus solutions, followed by infection in Vero cells in 12-well pates at final concentrations of 500, 100 or 20 pfu/well. After incubation at room temperature for 1-hour, different concentrations of GCV were added in the indicated wells and the cells were incubated at 37° C. 5% CO2. After 3 days of incubation, the plates were stained with 2% Crystal Violet and the number of plaques were counted. The above experiment was repeated 3 times.

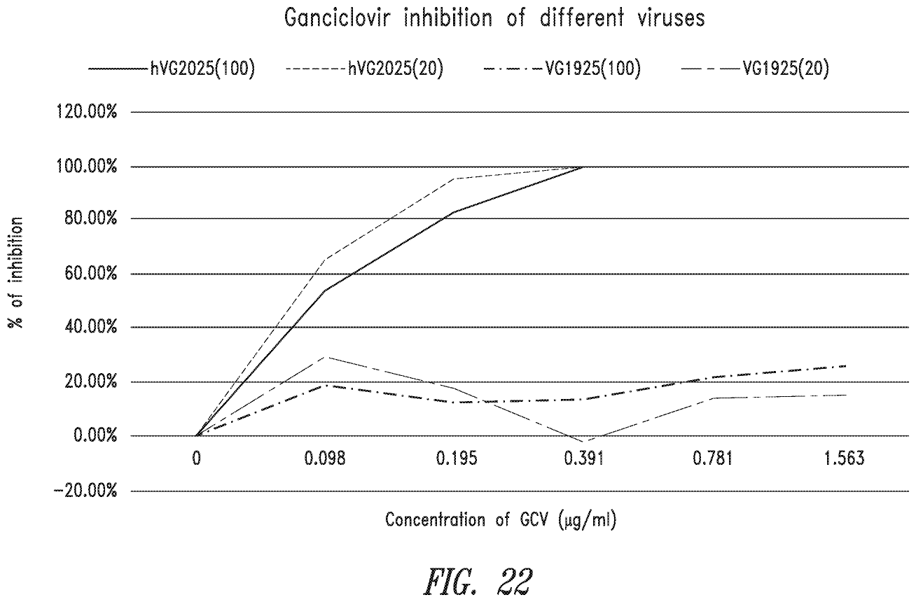

Results: The results are provided below in Tables 11 and 12 and in FIG. 22.

TABLE 11
GCV sensitivity of hVG2025

| hVG2025 #9 | | | | | | | % | |
|---|---|---|---|---|---|---|---|---|
| Virus | GCV | Plaque counting (pfu/well) Average | | | | | compare | |
| inoculated | concentration | Run | Run | Run | | | with GCV | % |
| (PFU/well) | (ug/ml) | 1 | 2 | 3 | Average | SD | 0 ug/ml | inhibition |
| 100 | 0 | 49 | 63.5 | 72.5 | 61.67 | 11.86 | 100.00% | 0.00% |
| | 0.098 | 19 | 32.5 | 33.5 | 28.33 | 8.1 | 45.95% | 54.05% |
| | 0.195 | 7 | 13 | 10.5 | 10.17 | 3.01 | 16.49% | 83.51% |
| | 0.391 | 0 | 0 | 0 | 0 | 0 | 0.00% | 100.00% |
| | 0.781 | 0 | 0 | 0 | 0 | 0 | 0.00% | 100.00% |
| | 1.563 | 0 | 0 | 0 | 0 | 0 | 0.00% | 100.00% |
| 20 | 0 | 13.5 | 14 | 16.5 | 14.67 | 1.61 | 100.00% | 0.00% |
| | 0.098 | 4 | 8 | 3.5 | 5.17 | 2.47 | 35.23% | 64.77% |
| | 0.195 | 0 | 0 | 2 | 0.67 | 1.15 | 4.55% | 95.45% |
| | 0.391 | 0 | 0 | 0 | 0 | 0 | 0.00% | 100.00% |
| | 0.781 | 0 | 0 | 0 | 0 | 0 | 0.00% | 100.00% |
| | 1.563 | 0 | 0 | 0 | 0 | 0 | 0.00% | 100.00% |

TABLE 12
GCV sensitivity of tk(−) VG1925

| VG1925 #2-1 (TK−) | | | | | | | % | |
|---|---|---|---|---|---|---|---|---|
| Virus | GCV | Plaque counting (pfu/well) Average | | | | | compare | |
| concentration | concentration | Run | Run | Run | | | with GCV | % |
| (PFU/well) | (ug/ml) | 1 | 2 | 3 | Average | SD | 0 ug/ml | inhibition |
| 100 | 0 | 77.5 | 87 | 119.5 | 94.67 | 22.02 | 100.00% | 0.00% |
| | 0.098 | 73 | 81 | 78 | 77.33 | 4.04 | 81.69% | 18.31% |
| | 0.195 | 65.5 | 82 | 100.5 | 82.67 | 17.51 | 87.32% | 12.68% |
| | 0.391 | 59 | 76 | 111 | 82 | 26.51 | 86.62% | 13.38% |
| | 0.781 | 60.5 | 69 | 94.5 | 74.67 | 17.69 | 78.87% | 21.13% |
| | 1.563 | 56.5 | 65 | 92 | 71.17 | 18.54 | 75.18% | 24.82% |
| 20 | 0 | 14.5 | 21 | 23 | 19.5 | 4.44 | 100.00% | 0.00% |
| | 0.098 | 13.5 | 18 | 10.05 | 13.85 | 3.99 | 71.03% | 28.97% |
| | 0.195 | 11.5 | 15 | 22 | 16.17 | 5.35 | 82.91% | 17.09% |
| | 0.391 | 13 | 26.5 | 20.5 | 20 | 6.76 | 102.56% | −2.56% |
| | 0.781 | 16 | 15.5 | 19 | 16.83 | 1.89 | 86.32% | 13.68% |
| | 1.563 | 13 | 17 | 20 | 16.67 | 3.51 | 85.47% | 14.53% |

Conclusions: The TK (+) hVG2025 is highly sensitive to Ganciclovir. The IC50 of GCV inhibits hVG2025 is <0.195 ug/ml and <0.39 ug/ml of GCV caused 100% virus inhibition. The above sensitivity to GCV by hVG2025 is in contrast to its parental strain VG1925 #2-1 (TK-) where GCV at 1.563 ug/ml (maximum concentration tested) could only cause ˜20% inhibition in virus replication.

Hence, the concentration required to inhibit hVG2025 is much lower than the clinical dose of Gancyclovir in humans (see e.g., Toxicity et al., 2017, Ganciclovir Injection [package insert]. Lenoir: EXELA Pharma Sciences, NC; 2017; where the clinical dose of Ganciclovir provides a Cmax in humans of 9 μg/ml).

### Example 16

### Virus Stability

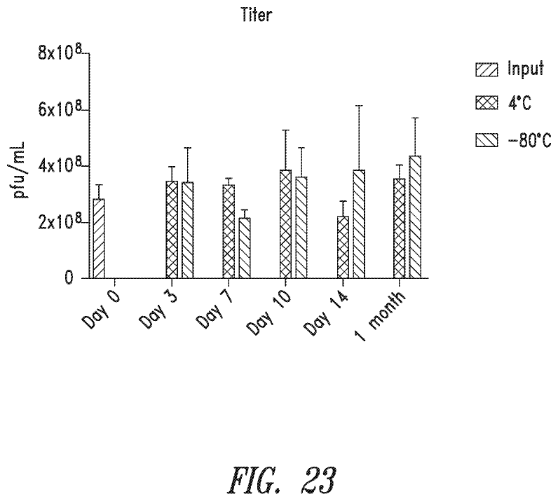

Stability data was collected using the pilot batches of VG2025 vials that were stored at 4° C. and −80° C., respectively, for up to 1 month. The titer of each was compared to the one before vialing. Under both temperatures, there is no significant virus titer loss for up to 1 month (FIG. 23).

### Example 17

Evaluation of Antitumor Efficacy of hVG2025 Virus in a Liver Cancer (Hep 3B-Luc) Model

Objective: The objective of this study was to evaluate the anti-tumor efficacy of VG2025 administered intravenously (i.v.) in the orthotopic human liver cancer xenograft model of Hep 3B-luc in female BALB/c nude mice.

Study design: The experimental design of the antitumor efficacy study of hVG2025 is summarized in Table 13.

TABLE 13
Efficacy Study Design

| | | | Dose | Dose | | |
|---|---|---|---|---|---|---|
| | | | (PFU/ | volume | | |
| Group | N | Treatment | mouse) | (μL) | Route | Schedule |
| 1 | 8 | Vehicle | — | 100 | i.v. | Dosed at PG-D1 |
| 2 | 8 | VG2025 | 2.40E+04 | 100 | i.v. | Dosed at PG-D1, |
| | | | | | | PG-D9, PG-D11, |
| | | | | | | PG-D13 |
| 3 | 8 | VG2025 | 2.40E+05 | 100 | i.v. | Dosed at PG-D1, |
| | | | | | | PG-D9, PG-D11, |
| | | | | | | PG-D13 |
| 4 | 8 | VG2025 | 2.40E+06 | 100 | i.v. | Dosed at PG-D1 |
| 5 | 8 | VG2025 | 2.40E+07 | 100 | i.v. | Dosed at PG-D1 |

N: number of animals per group
Dose volume: dosing volume was 100 μL/mouse

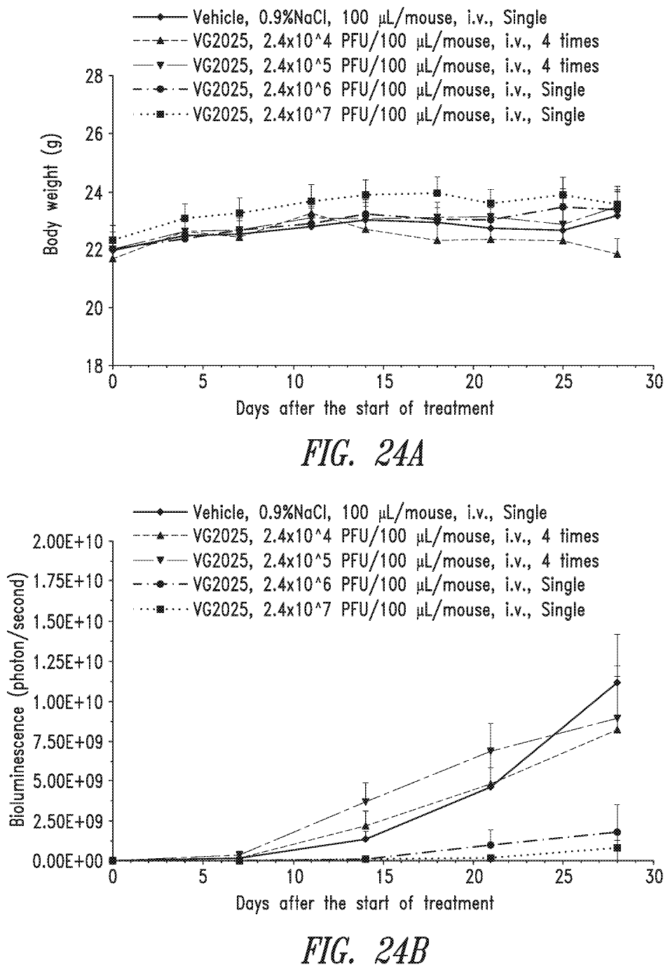

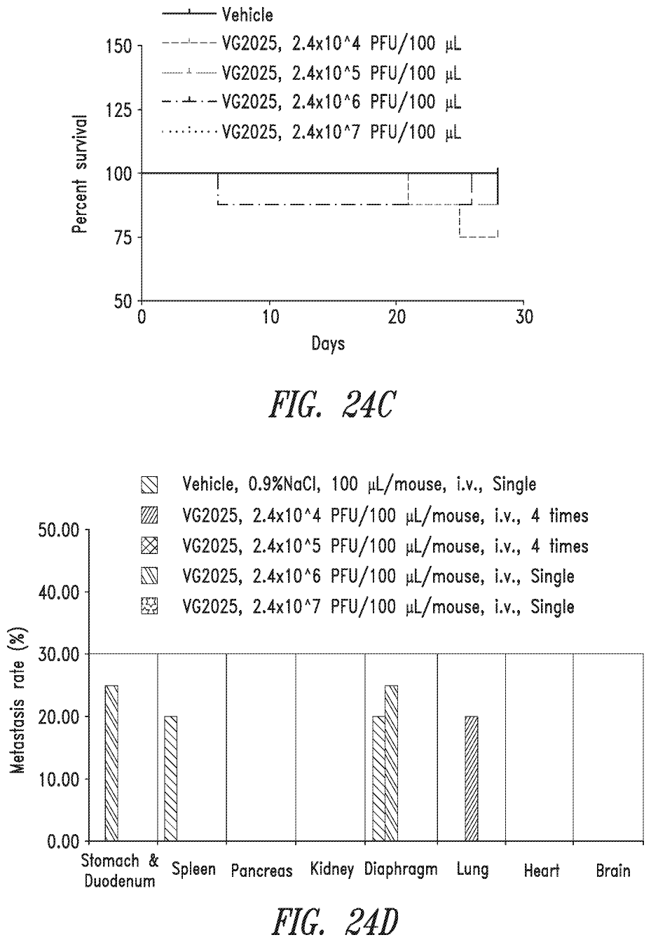

Results: Body weight changes after administering VG2025 to female BALB/c nude mice bearing orthotopic Hep 3B-luc established tumors are shown in FIG. 24A. Data points represent group mean body weight. Error bars represent standard error of the mean (SEM).

The mean bioluminescence over time in female BALB/c nude mice bearing orthotopic Hep 3B-luc xenografts dosed with VG2025 is shown in Table 14 and FIG. 24B.

TABLE 14
Mean Bioluminescence over Time (×107 photon/second)^{a}

| | | VG2025 | VG2025 | VG2025 | VG2025 |
|---|---|---|---|---|---|
| | | (2.4 × 10\hat{ }4 PFU/ | (2.4 × 10\hat{ }5 PFU/ | (2.4 × 10\hat{ }6 PFU/ | (2.4 × 10\hat{ }7 PFU/ |
| Treatment | Vehicle | 100 μL/mouse) | 100 μL/mouse) | 100 μL/mouse) | 100 μL/mouse) |
| 0 | 2 ± 1 | 2 ± 1 | 2 ± 1 | 2 ± 1 | 2 ± 1 |
| 7 | 20 ± 6 | 21 ± 10 | 42 ± 13 | 2 ± 1 | 2 ± 1 |
| 14 | 134 ± 52 | 220 ± 91 | 373 ± 117 | 12 ± 9 | 5 ± 2 |
| 21 | 468 ± 121 | 487 ± 189 | 689 ± 171 | 103 ± 100 | 23 ± 12 |
| 28 | 1120 ± 297 | 830 ± 390 | 901 ± 253 | 182 ± 176 | 84 ± 53 |

^{a}Mean +/− SEM, n = 8

The survival curve after administering VG2025 to female BALB/c nude mice bearing orthotopic Hep 3B-luc established tumors is shown in FIG. 24C.

The metastasis rates of these mice are shown in Table 15 and FIG. 24D. Bioluminescence intensity was calculated based on euthanized animals detected by IVIS machine. No significant metastases was detected in any group.

TABLE 15
Metastasis Rate (%)

| | | VG2025 | VG2025 | VG2025 | VG2025 |
|---|---|---|---|---|---|
| Metastasis | | (2.4 × 10\hat{ }4 PFU/ | (2.4 × 10\hat{ }5 PFU/ | (2.4 × 10\hat{ }6 PFU/ | (2.4 × 10\hat{ }7 PFU/ |
| rate (%) | Vehicle | 100 μL/mouse) | 100 μL/mouse) | 100 μL/mouse) | 100 μL/mouse) |
| Stomach and | 0 | 25 | 0 | 0 | 0 |
| Duodenum | | | | | |
| Spleen | 20 | 0 | 0 | 0 | 0 |
| Pancreas | 0 | 0 | 0 | 0 | 0 |
| Kidney | 0 | 0 | 0 | 0 | 0 |
| Diaphragm | 20 | 25 | 0 | 0 | 0 |
| Lung | 0 | 0 | 20 | 0 | 0 |
| Heart | 0 | 0 | 0 | 0 | 0 |
| Brain | 0 | 0 | 0 | 0 | 0 |

Conclusion: The anti-tumor effects of intravenously delivered hVG2025 was confirmed in this mouse model xenografted with human liver cancer cells. Compared to the vehicle control group, treatment with VG2025 (2.4×10\hat{ }6 PFU/100 μl/mouse) and VG2025 (2.4×10\hat{ }7 PFU/100 μL/mouse) showed obvious anti-tumor effects in this model of liver cancer. Significantly, there no significant metastases were observed in any treatment group.

### Example 18

Evaluation of Antitumor Efficacy of mVG2025 in a B Cell Lymphoma (A20-Luc) Model

Objective: The objective of this study was to evaluate the anti-tumor efficacy of VG2025 administered intravenously (i.v.) in the orthotopic human B cell lymphoma xenograft model in female BALB/c nude mice.

Study design: Before inoculation with the lymphoma cells, mice (8 per group) were pre-immunized with two subcutaneous injections of mVG2025 at 10\hat{ }6PFU/mouse. Mice were then inoculated with A20-Luc B cell lymphoma cells intravenously. Details of the experimental design are set forth in Table 16.

TABLE 16
Efficacy Study Design

| | | | Dose | Dose | | |
|---|---|---|---|---|---|---|
| | | | (PFU/ | volume | | |
| Group | N | Treatment | mouse) | (μL) | Route | Schedule |
| 1 | 8 | Vehicle | — | 100 | i.v. | Single |
| 2 | 8 | mVG2025 | 2.40E+05 | 100 | i.v. | Single |
| 3 | 8 | mVG2025 | 2.40E+06 | 100 | i.v. | Single |
| 4 | 8 | mVG2025 | 2.40E+07 | 100 | i.v. | Single |
| 54 | 8 | mVG2025 | 2.40E+07 | 100 | i.v. | Single |

N: number of animals per group
Dose volume: dosing volume was 100 μL/mouse

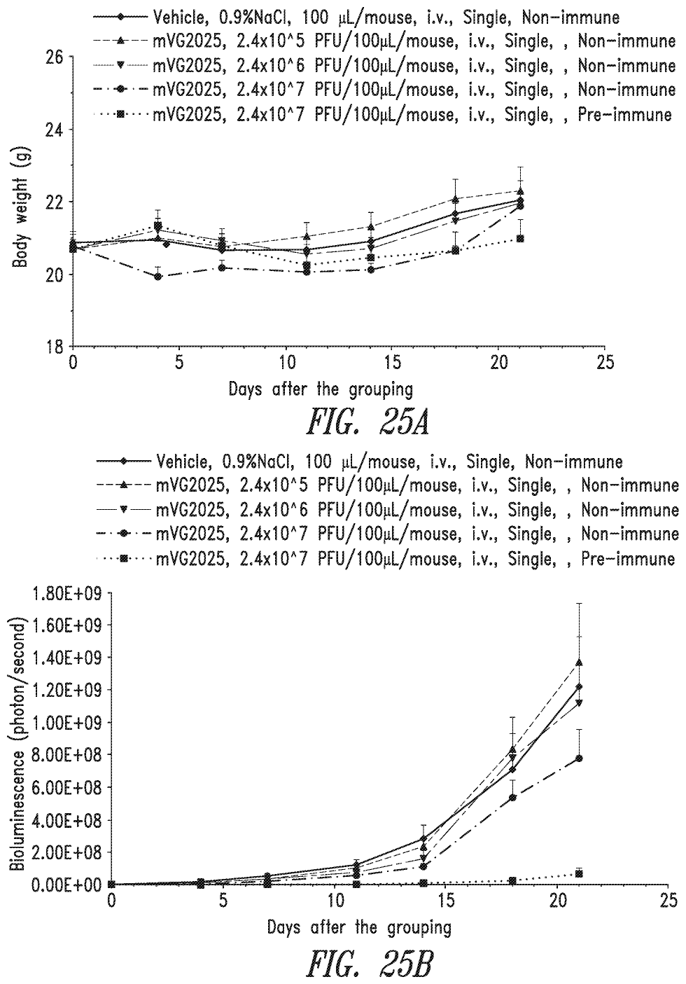

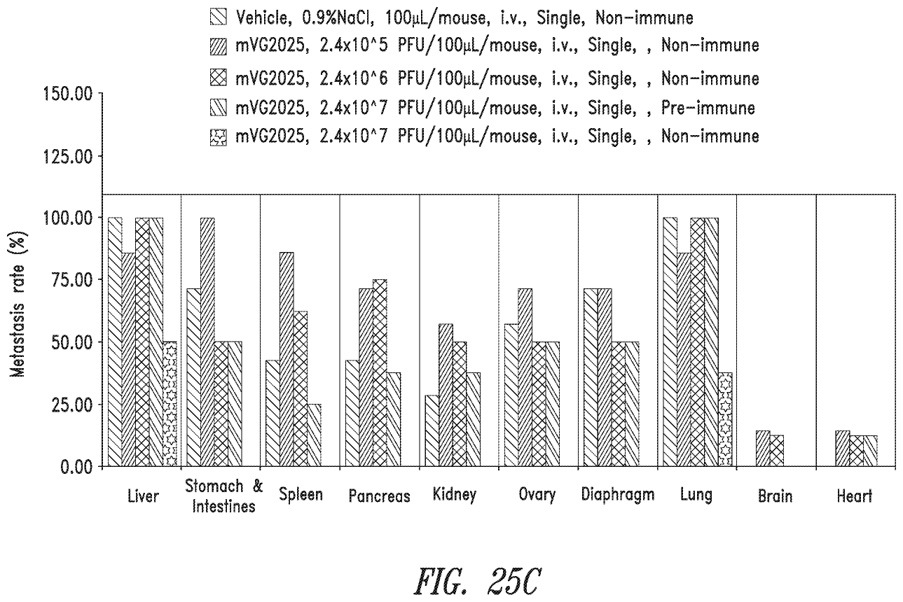

Results: The body weight changes after administering mVG2025 to female BALB/c mice bearing orthotopic A20-luc established tumors are shown in FIG. 25A. Data points represent group mean body weight. Error bars represent standard error of the mean (SEM).

The mean bioluminescence over time in female BALB/c mice bearing orthotopic A20-luc xenograft tumors dosed with mVG2025 is shown in Table 17 and FIG. 25B.

TABLE 17
Mean Bioluminescence over Time (×10⁶ photon/second

| | | mVG2025 | mVG2025 | mVG2025 | mVG2025 |
|---|---|---|---|---|---|
| | | (2.4 × 10\hat{ }5 PFU/ | (2.4 × 10\hat{ }6 PFU/ | (2.4 × 10\hat{ }7 PFU/ | (2.4 × 10\hat{ }7 PFU/ |
| | | 100 μL/mouse) | 100 μL/mouse) | 100 μL/mouse) | 100 μL/mouse) |
| Treatment | Vehicle | Non-immune | Non-immune | Non-immune | Pre-immune |
| 0 | 1.8 ± 0.1 | 1.8 ± 0.1 | 1.8 ± 0.2 | 1.8 ± 0.1 | 1.8 ± 0.1 |
| 4 | 23.7 ± 5.9 | 12.3 ± 2.8 | 8.2 ± 2.1 | 3.7 ± 0.7 | 2.7 ± 0.8 |
| 7 | 55.9 ± 14.3 | 37.2 ± 10.4 | 35.9 ± 11.7 | 22.1 ± 4.80 | 2.5 ± 0.6 |
| 11 | 126.2 ± 30.6 | 97.8 ± 29.6 | 76.4 ± 20.3 | 59.3 ± 9.5 | 3.9 ± 1.1 |
| 14 | 289.3 ± 76.7 | 232.0 ± 58.0 | 161.7 ± 50.1 | 110.7 ± 16.9 | 10.8 ± 4.8 |
| 18 | 708.6 ± 220.4 | 843.1 ± 162.2 | 783.6 ± 249.6 | 540.2 ± 105.1 | 23.6 ± 10.9 |
| 21 | 1224.6 ± 308.4 | 1375.5 ± 356.1 | 1118.6 ± 259.8 | 780.9 ± 176.3 | 68.1 ± 33.5 |

The metastasis rates observed in this study are shown in Table 18 and FIG. 25C. Bioluminescence intensity was calculated based on euthanized animals detected by IVIS machine.

TABLE 18
Metastasis Rate (%)

| | | mVG2025 | mVG2025 | mVG2025 | mVG2025 |
|---|---|---|---|---|---|
| | | (2.4 × 10\hat{ }5 PFU/ | (2.4 × 10\hat{ }6 PFU/ | (2.4 × 10\hat{ }7 PFU/ | (2.4 × 10\hat{ }7 PFU/ |
| Metastasis | | 100 μL/mouse) | 100 μL/mouse) | 100 μL/mouse) | 100 μL/mouse) |
| rate (%) | Vehicle | Non-immune) | Non-immune) | Non-immune) | Pre-immune) |
| Liver | 100.0 | 85.7 | 100.0 | 100.0 | 50.0 |
| Stomach and | 71.4 | 100.0 | 50.0 | 50.0 | 0.0 |
| intestines | | | | | |
| Spleen | 42.9 | 85.7 | 62.5 | 25.0 | 0.0 |
| Pancreas | 42.9 | 71.4 | 75.0 | 37.5 | 0.0 |
| Kidney | 28.6 | 57.1 | 50.0 | 37.5 | 2.0 |
| Ovary | 57.1 | 71.4 | 50.0 | 50.0 | 0.0 |
| Diaphragm | 71.4 | 71.4 | 50.0 | 50.0 | 0.0 |
| Lung | 100.0 | 85.7 | 100.0 | 100.0 | 37.5 |
| Brain | 0.0 | 14.3 | 12.5 | 0.0 | 0.0 |
| Heart | 0.0 | 14.3 | 12.5 | 12.5 | 0.0 |

Conclusion: In this study, the therapeutic efficacy of mVG2025 was evaluated in an orthotopic A20-luc B cell lymphoma xenograft model. Compared to the control (vehicle) group, treatment with mVG2025 (at 2.4×10\hat{ }7 PFU/100 μL/mouse, pre-immune) showed obvious anti-tumor activity in this model of B cell lymphoma.

### Example 19

Evaluation of Antitumor Efficacy of Low Dose mVG2025 Virus in B Cell Lymphoma (A20-Luc) Model

Objective: The objective of this study was to evaluate the anti-tumor efficacy of VG2025 administered intravenously (i.v.) in the orthotopic human B cell lymphoma xenograft model in female BALB/c nude mice.

Study design: Before inoculation with lymphoma cells, mice were pre-immunized with two subcutaneous injections of mVG2025 at 10\hat{ }6PFU/mouse. Mice were then inoculated with A20-Luc B cell lymphoma cells intravenously. Details of the experimental design are set forth in Table 19.

TABLE 19
Efficacy Study Design

| | | | Dose | Dose | | |
|---|---|---|---|---|---|---|
| | | | (PFU/ | volume | | |
| Group | N | Treatment | mouse) | (μL) | Route | Schedule |
| 1 | 8 | Vehicle | — | 100 | i.v. | Single |
| 2 | 8 | mVG2025 | 1.00E+05 | 100 | i.v. | Single |
| 3 | 8 | mVG2025 | 1.00E+06 | 100 | i.v. | Single |
| 4 | 8 | mVG2025 | 1.00E+05 | 100 | i.v. | Single |
| 5 | 8 | mVG2025 | 1.00E+06 | 100 | i.v. | Single |

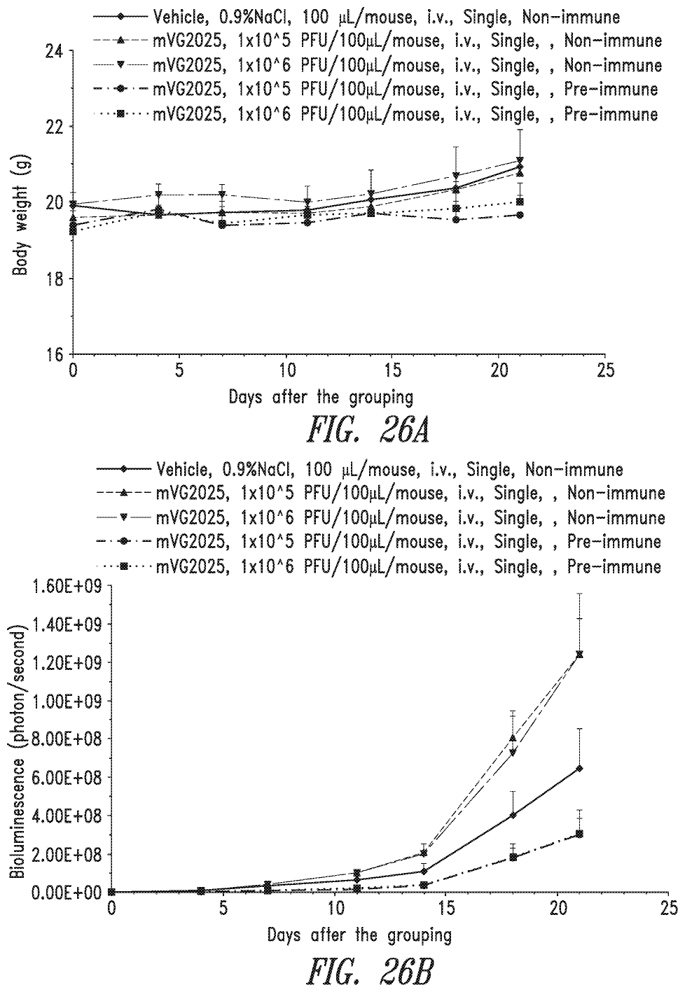

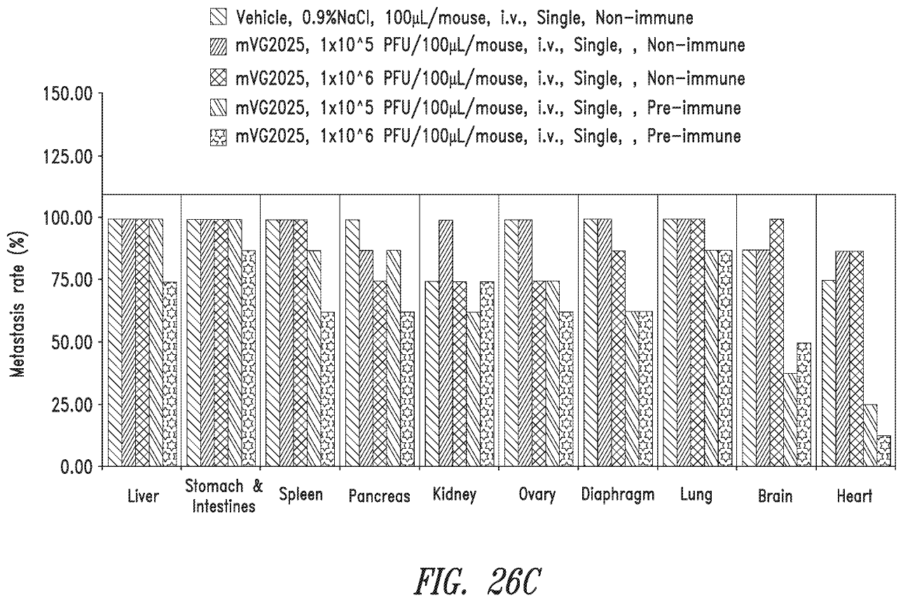

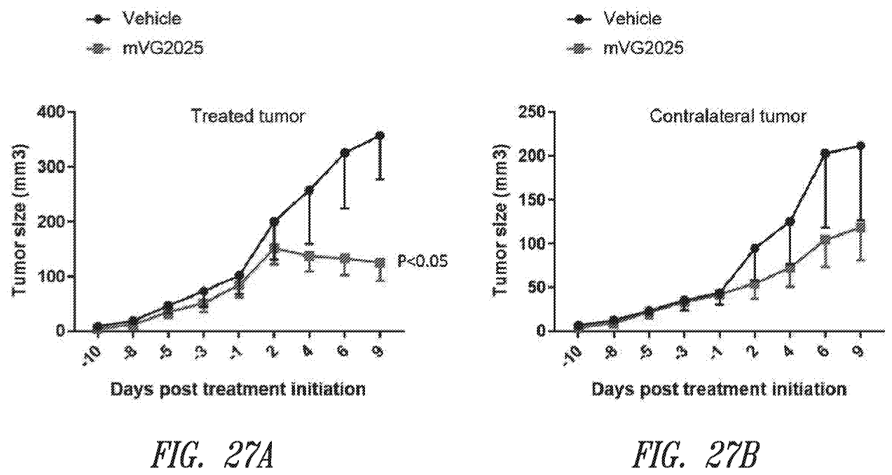

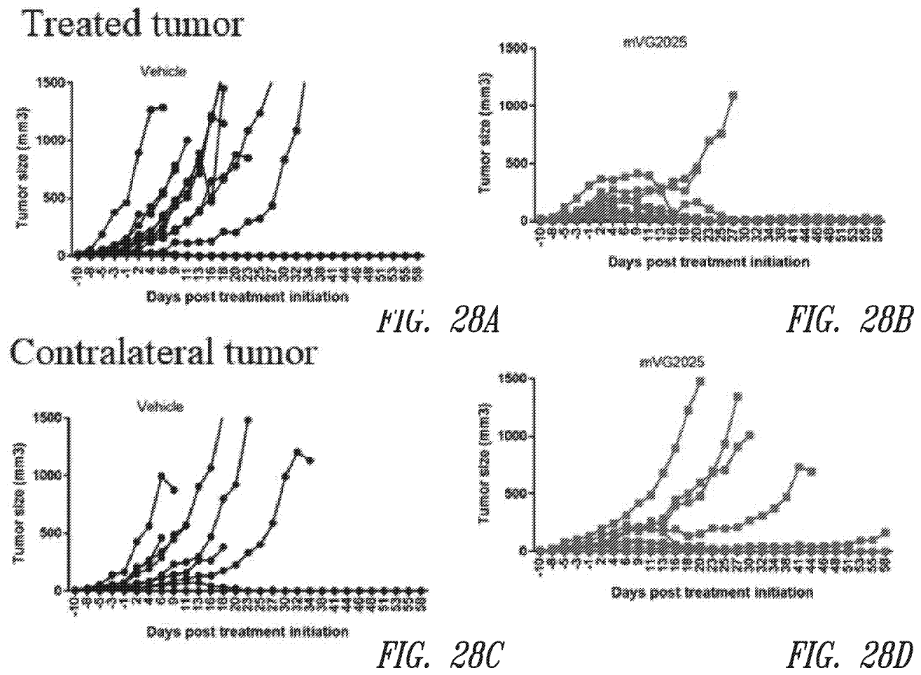

Results: The body weight changes after administering mVG2025 to female BALB/c mice bearing orthotopic A20-luc established tumors are shown in FIG. 26A. Data points represent group mean body weight. Error bars represent standard error of the mean (SEM).

The mean bioluminescence over time in female BALB/c mice bearing orthotopic A20-luc xenograft tumors dosed with mVG2025 is shown in Table 20 and FIG. 26B.

TABLE 20
Mean Bioluminescence over Time (×10⁶ photon/second)

| | | mVG2025 | mVG2025 | mVG2025 | mVG2025 |
|---|---|---|---|---|---|
| | | (1.0 × 10\hat{ }5 PFU/ | (1.0 × 10\hat{ }6 PFU/ | (1.0 × 10\hat{ }5 PFU/ | (1.0 × 10\hat{ }6 PFU/ |
| | | 100 μL/mouse) | 100 μL/mouse) | 100 μL/mouse) | 100 μL/mouse) |
| | Vehicle | Non-immune | Non-immune | Pre-immune | Pre-immune |
| 0 | 1.36 ± 0.051 | 1.36 ± 0.05 | 1.36 ± 0.11 | 1.60 ± 0.09 | 1.60 ± 0.17 |
| 4 | 7.09 ± 1.34 | 9.97 ± 1.52 | 7.78 ± 1.57 | 6.49 ± 0.92 | 6.69 ± 1.48 |
| 7 | 33.53 ± 7.34 | 43.21 ± 9.39 | 41.15 ± 8.92 | 9.85 ± 2.49 | 9.28 ± 2.62 |
| 11 | 66.58 ± 25.84 | 103.26 ± 18.24 | 99.81 ± 23.43 | 15.94 ± 5.26 | 23.03 ± 10.71 |
| 14 | 108.95 ± 40.83 | 208.17 ± 37.84 | 198.82 ± 55.72 | 36.31 ± 12.39 | 38.82 ± 16.39 |
| 18 | 403.03 ± 121.77 | 806.50 ± 137.66 | 723.5 ± 195.14 | 178.59 ± 52.42 | 186.4 ± 65.57 |
| 21 | 647.68 ± 205.44 | 1235.31 ± 191.10 | 1240.76 ± 319.15 | 307.81 ± 73.77 | 307.84 ± 117.85 |

The metastasis rates are shown in Table 21 and FIG. 26C.

TABLE 21
Metastatsis Rate (%)

| | | mVG2025 | mVG2025 | mVG2025 | mVG2025 |
|---|---|---|---|---|---|
| | | (2.4 × 10\hat{ }5 | (2.4 × 10\hat{ }6 | (2.4 × 10\hat{ }7 | (2.4 × 10\hat{ }7 |
| | | PFU/ | PFU/ | PFU/ | PFU/ |
| Metastasis | | 100 μL/mouse | 100 μL/mouse | 100 μL/mouse | 100 μL/mouse |
| rate (%) | Vehicle | Non-immune) | Non-immune) | Pre-immune) | Immune) |
| Liver | 100.00 | 100.00 | 100.00 | 100.00 | 75.00 |
| Stomach and | 100.00 | 100.00 | 100.00 | 100.00 | 87.50 |
| intestines | | | | | |
| Spleen | 100.00 | 100.00 | 100.00 | 87.50 | 62.50 |
| Pancreas | 100.00 | 87.50 | 75.00 | 87.50 | 62.50 |
| Kidney | 75.00 | 100.00 | 75.00 | 62.50 | 75.00 |
| Ovary | 100.00 | 100.00 | 75.00 | 75.00 | 62.50 |
| Diaphragm | 100.00 | 100.00 | 87.50 | 62.50 | 62.50 |
| Lung | 100.00 | 100.00 | 100.00 | 87.50 | 87.50 |
| Brain | 87.50 | 87.50 | 100.00 | 37.50 | 50.00 |
| Heart | 75.00 | 87.50 | 87.50 | 25.00 | 12.50 |

Conclusion: In this study, the therapeutic efficacy of mVG2025 was evaluated in an orthotopic A20-luc B cell lymphoma xenograft model. Compared to the control (vehicle) group, treatment with mVG2025 (at 2.4×10\hat{ }6 PFU/100 μL/mouse, pre-immune) and mVG2025 (at 2.4×10\hat{ }5 PFU/100 μL/mouse, pre-immune) showed obvious anti-tumor activity in this model of B cell lymphoma.

### Example 20

Evaluation of hVG2025 Acute Toxicity in Primates

The study was conducted to assess the acute toxicity of hVG2025, after a single dose administration in Rhesus monkey via subcutaneous injection or intravenous injection.

Male and female Rhesus monkeys were administered 0 PFU/kg, 1.0×10⁹ PFU/kg via subcutaneous administration, and 2.0×10⁹ PFU/kg via intravenous administration as single dose. Six Rhesus monkeys were randomly assigned into 3 groups (1 animal/sex/group), the infusion rate of intravenous administration was 2 ml/min, and the dose volume was 5 ml/kg and 10 ml/kg of subcutaneous administration and intravenous administration, respectively.

The following parameters and endpoints were assessed: morbidity and mortality; clinical observations; body weights; food consumption; body temperature; electrocardiogram examination; hematology and coagulation; clinical chemistry; immune function and gross pathology.

All animals survived to the scheduled sacrifice. No test article-related effects on clinical observation, body weights, food consumption, body temperature, electrocardiograms examination, hematology and coagulation, clinical chemistry, immune function or gross pathology were noted.

Conclusion: male and female Rhesus monkeys were administered 0 PFU/kg, 1.0 ×10⁹ PFU/kg via subcutaneous administration, and 2.0×10⁹ PFU/kg via intravenous administration as single dose. All animals survived to the scheduled sacrifice. No hVG2025-related abnormal changes in clinical observation, body weight, food consumption, body temperature, electrocardiograms examination, hematology, coagulation, clinical chemistry, immune function or gross pathology were noted. Therefore, the maximal tolerated dose (MTD) of hVG2025 in Rhesus Monkeys via subcutaneous injection was determined to be 1.0×10⁹ PFU/kg. The maximal tolerated dose (MTD) of hVG2025 in Rhesus Monkeys via intravenous injection was determined to be 2.0×10⁹ PFU/kg.

### Example 21

Abscopal Antitumor Efficacy of mVG2025 in Murine Colon Cancer (CT26) Mouse Model

Objective: The experimental goal was to determine the efficacy and safety of mVG2025, which is the surrogate version of hVG2025 expressing murine IL-12, in a dual CT26 syngeneic mouse colon cancer model incorporating both a primary and a secondary tumor inoculated into opposing mouse flanks.

Study design: 24 female SPF-grade BALB/c mice were subcutaneously injected twice with 5×10\hat{ }5 CT26 cells per mouse, once into each flank, and randomized into two groups with 12 mice in each group. Group 1 was vehicle control which was intratumorally injected with PBS. Group 2 was the test group, administered with 5 doses on 5 consecutive days of mVG2025 at 1×10\hat{ }8 PFU/mouse for each dose via intratumoral injection. Injections were performed into a single tumor per mouse, with the second tumor on the opposing flank remaining uninjected. All animals were appropriately identified by marking on different body parts, housed, fed according to standard protocols.

Measurements: All mice were observed at least twice daily after administration for clinical symptoms. Mice body weight and tumor size were measured three times a week. Tumor volumes were measured using a caliper (Length×Width×Depth×0.5236).

Results: Statistically significant tumor growth inhibition was observed following five consecutive intratumoral treatments with mVG2025 at 9 days post-treatment initiation compared to the vehicle control group (FIG. 27A). Tumor growth inhibition on the contralateral abscopal untreated tumor did not reach statistical significance at the same timepoint, although average tumor size was observed to trend downwards in the treated mice compared to the control group (FIG. 27B). At 30 days post-treatment initiation, 7 out of 12 mice in the mVG2025 treated group showed a complete response as evidenced by complete regression of both virus-treated and contralateral abscopal tumors. By contrast, 10 out of 12 control group mice were euthanized due to tumor burden by 34 days post-treatment initiation (see FIGS. 28A, 28B, 28C and 28D).

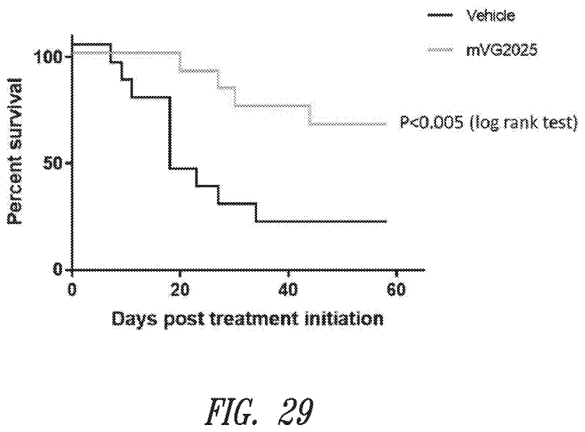

Mice treated with mVG2025 exhibited a statistically significant increase in percent survival compared to mice treated with vehicle control. Specifically, 8 of 12 mice treated with mVG2025 survived until the experimental endpoint (58 days post treatment initiation). On the other hand, 10 of 12 mice in the control group reached a humane endpoint due to tumor burden prior to reaching the experimental endpoint (FIG. 29).

Conclusions: The abscopal anti-tumor immune efficacy and survival benefit of treatment with mVG2025 was confirmed in a syngeneic dual CT26 murine colon cancer model, resulting in complete clearance of implanted tumor from both the injected and noninjected sides in 7 out of 12 mice. Moreover, no clinical signs of HSV-1 related toxicity were observed.

### Example 22

Abscopal Antitumor Efficacy of mVG2025 in Murine B-Cell Lymphoma (A20) Mouse Model

Objective: The experimental goal was to determine the efficacy and safety of mVG2025, which is the surrogate version of hVG2025 expressing murine IL-12, in a dual A20 syngeneic mouse B-cell lymphoma model incorporating both a primary and a secondary tumor inoculated into opposing mouse flanks.

Study design: 19 SPF-grade BALB/c mice were subcutaneously injected twice with 2.5×10\hat{ }6 A20 cells per mouse, once into each flank, and randomized into two groups. Group 1 was the vehicle control which consisted of 9 mice intratumorally injected with PBS. Group 2 was the test group, consisting of 10 mice administered with 5 doses on 5 consecutive days of mVG2025 at 1×10\hat{ }8 PFU/mouse for each dose via intratumoral injection. Injections were performed into a single tumor per mouse, with the second tumor on the opposing flank remaining uninjected. All animals were appropriately identified by marking on different body parts, housed, fed according to standard protocols.

Measurements: All mice were observed at least twice daily after administration for clinical symptoms. Mice body weight and tumor size were measured three times a week. Tumor volumes were measured using a caliper (Length×Width×Depth×0.5236).

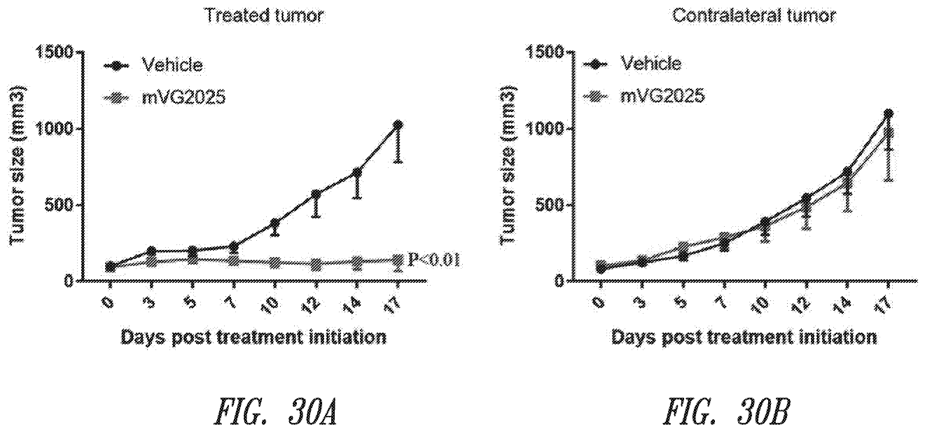

Results: Statistically significant tumor growth inhibition was observed for the treated tumors following five consecutive intratumoral treatments with mVG2025 at 17 days post-treatment initiation compared to the vehicle control group (FIG. 30A). Tumor growth inhibition on the contralateral untreated tumor did not reach statistical significance at the same timepoint (FIG. 30B), although by 75 days post-treatment initiation, 4 out of 10 mice in the mVG2025 treated group showed a complete response as evidenced by complete regression of both virus-treated and contralateral abscopal tumors. By contrast, all control group mice were euthanized due to tumor burden by 42 days post-treatment initiation (see FIGS. 31A, 31B, 31C and 31D).

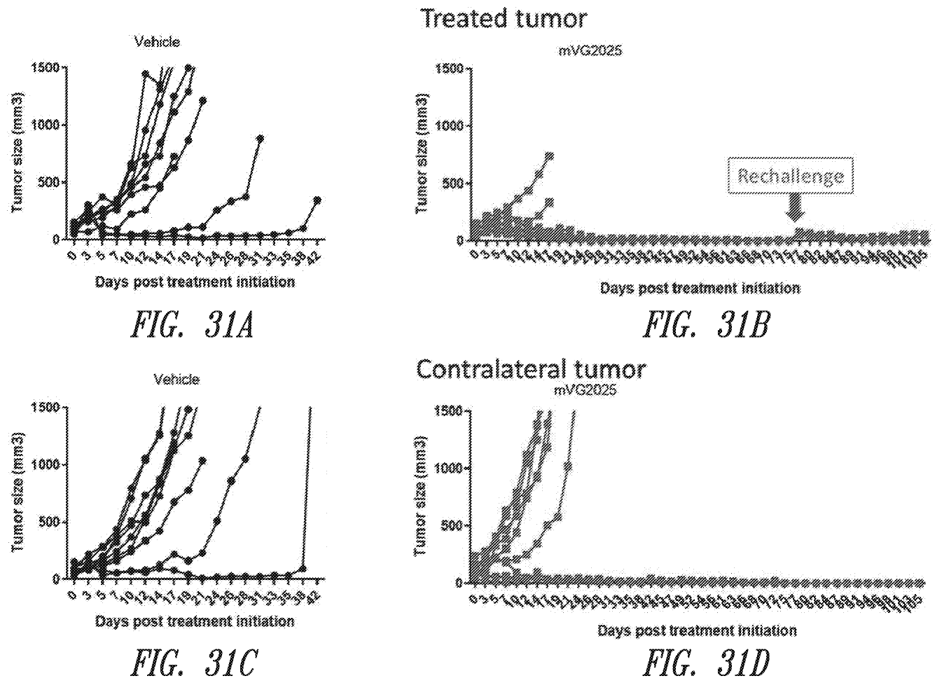

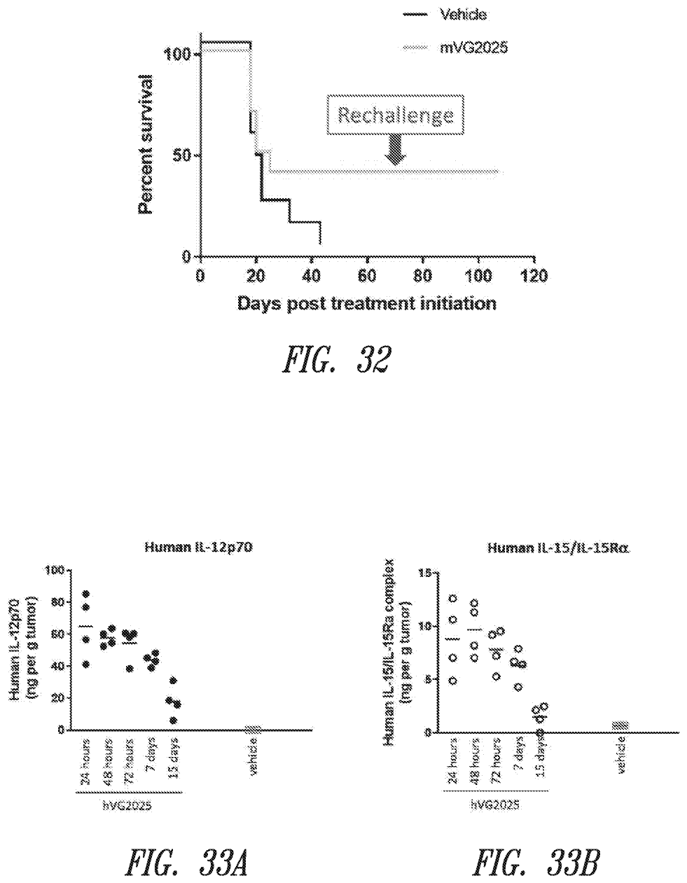

To further demonstrate that the four tumor-free mice treated with mVG2025 were able to generate an anti-tumor immune response, they were re-challenged with A20 tumor cells at 77 days after the initial treatment. The newly established tumors slowly regressed in 3 of 4 mice by 10⁷ days post-treatment initiation without further mVG2025 treatment (FIG. 32) which suggests the presence of an immune response against A20 tumor cells.

4 out of 10 mice treated with mVG2025 survived until the experimental endpoint (10⁷ days post treatment initiation). On the other hand, all control group mice reached a humane endpoint due to tumor burden by 42 days post treatment initiation.

Conclusions: The abscopal anti-tumor immune efficacy and survival benefit of treatment with mVG2025 was confirmed in a syngeneic dual A20 murine B-cell lymphoma model, resulting in complete clearance of implanted tumor from both the injected and noninjected sides in 4 out of 10 mice. The presence of anti-tumor immune memory was further demonstrated in the 4 mice with a complete response by re-challenging them with A20 tumor cells, resulting in complete tumor clearance in 3 out of the 4 mice. Moreover, no clinical signs of HSV-1 related toxicity were observed.

### Example 23

### Intratumoral and Systemic Detection of IL-12 and IL-15 Payload

Objective: The experimental goal was to determine the payload level of intratumorally delivered hVG2025 virus in a human lung cancer (A549) xenograft mouse model.

Study design: 23 female SPF-grade athymic nude mice were subcutaneously injected with 2.5×10\hat{ }6 A549 cells per mouse and randomized into two groups. Group 1 was vehicle control which consisted of 3 mice that were intratumorally injected with 7.5% glycerol dissolved in PBS. Group 2 was the test group consisting of 20 mice which were administered a single dose of hVG2025 at 5×10\hat{ }7 PFU/mouse via intratumoral injection. All vehicle-injected mice were euthanized 2 days post-treatment, while 4 of the virus-injected mice were euthanized at 1 day, 2 days, 3 days, 7 days, and 14 days post-treatment. Tumor and blood serum were harvested from each mouse and used for ELISA to detect IL-12 and IL-15/IL-15RA.

Measurements: Mice body weight and tumor size were measured three times a week. Tumor volumes were measured using a caliper (Length×Width×Depth×0.5236). Whole tumors were harvested and immediately snap frozen after humanely euthanizing the animals. Blood samples were also collected to extract serum. Both tumor and serum were subjected to ELISA assay to determine IL-12 and IL-15 concentration in accordance with the protocol provided by the vendor of the ELISA kit.

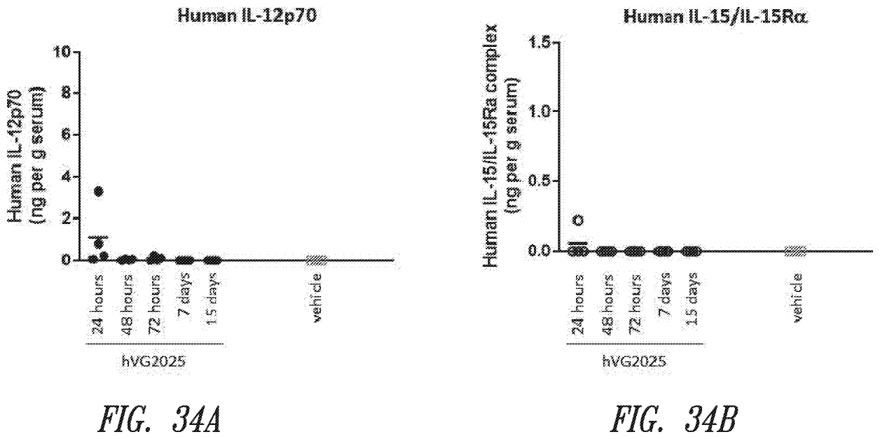

Results: A549 tumor and serum samples were collected from nude mice intratumorally injected with either hVG2025 or vehicle control. Kinetics of human IL-12p70 and human IL-15/IL-15Ra complex production in tumor tissue (FIGS. 33A and 33B) and serum (FIGS. 34 A and 34B) were analyzed using ELISA assay at 24 h, 48 h, 72h, 7d, and 15d post injection. In tumor tissue, detection of human IL-12p70 and human IL-15/IL-15Ra peaked at 24 hours after hVG2025 injection and remained detectable until 15 days after injection. Human IL-12p70 and human IL-15/IL-15Ra were detectable in serum samples at the 24-hour time point, but at less than 1% of the concentration detected in tumor tissue, and they rapidly dropped to undetectable levels in serum at subsequent timepoints.

Conclusions: The vast majority of human IL-12p70 and human IL-15/IL-15Rx was localized to the tumor with no evidence of systemic release, showing that payload leakage from intratumorally injected hVG2025 is not a safety concern.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The following are some exemplary numbered embodiments of the present disclosure.

1. A recombinant herpes virus comprising a modified oncolytic herpes virus genome, wherein the modified herpes virus genome comprises at least one miRNA target sequence operably linked to a first copy of an ICP34.5 gene, and a second copy of the ICP34.5 gene comprises an inactivating mutation. Within certain embodiments the recombinant herpes virus is a recombinant herpes simplex virus (such as HSV-1 or HSV-2).

2. The recombinant herpes virus of embodiment 1, wherein the mutation is deletion of at least one terminal repeat long (R_{L}) region or deletion of the terminal repeat long region and a deletion of the terminal repeat short (R_{S}) region of the viral genome. Examples include a deletion of the terminal repeat long (R_{L}) region of the viral genome which encodes one copy of the genes functionally equivalent to HSV-1 ICPO and ICP34.5, or a deletion of the terminal repeat long (R_{L}) region and a deletion of the terminal repeat short (R_{S}) region of the viral genome which encodes one copy of the gene functionally equivalent to HSV-1 ICP4. Within other embodiments the mutation may comprise at least one deletion of an internal repeat long region, or a deletion of an internal repeat long region and an internal repeat short region. Within other embodiments the mutation may comprise a deletion of one repeat long (R_{L}) region, or a deletion of one repeat long (R_{L}) region and a deletion of one repeat short (R_{S}) region of the viral genome. Within preferred embodiments of the invention the deletion is of one terminal repeat long region alone and has enhanced stability upon passage as compared to an HSV with deletions in both the terminal repeat long region and the terminal repeat short region. Within optional embodiments the mutation is a deletion containing the second copy of the ICP34.5 gene.

3. The recombinant herpes virus according to any one of embodiments 1 or 2, wherein the herpes virus is a Herpes simplex virus, and further comprising from two to ten miRNA target sequences operably linked to the first copy of the ICP34.5 gene.

4. The recombinant herpes simplex virus according to any one of embodiments 1, 2, or 3, wherein the miRNA target sequences are inserted into a 3′ untranslated region of the first copy of the ICP34.5 gene. Within a further embodiments the miRNA target sequences are inserted in tandem into the 3′ untranslated region. Within various embodiments, identical, or, varying lengths of linker DNA can be inserted between different miRNA binding sites. Within certain embodiments the linkers range from 1 to 50 base pairs. Within other embodiments the linker is less than 10 base pairs.

5. The recombinant herpes simplex virus according to any one of embodiments 1, 2, 3 or 4, wherein the from two to ten miRNA target sequences bind at least two different miRNAs.

6. The recombinant herpes simplex virus according to any one of embodiments 1, 2, 3, 4, or 5, wherein the miRNAs target sequences target an miRNA selected from the group consisting of miR-124, miR-124*, and miR-143.

7. The recombinant herpes virus according to any one of embodiments 1, 2, 3, 4, 5, or 6, wherein the herpes virus is Herpes simplex virus and wherein the modified herpes virus genome comprises additional mutations or modifications in viral genes ICP4 and/or ICP27.

8. The recombinant herpes virus according to any one of embodiments 1, 2, 3, 4, 5, 6, or 7, wherein the modification comprises replacing a native viral promoter with a tumor specific promoter.

9. The recombinant herpes virus according to any one of embodiments 1, 2, 3, 4, 5, 6, 7, or 8, wherein the modification is replacement of the entire promoter-regulatory region of ICP4 or ICP27, optionally, with a tumor specific promoter.

10. The recombinant herpes virus according to any one of embodiments, 1, 2, 3, 4, 5, 6, 7, 8, or 9, wherein the ICP27 promoter is replaced with a hCEA promoter.

11. The recombinant herpes virus according to any one of embodiments 1, 2, 3, 4, 5, 6, 7, 8, 9, or 10, further comprising at least one nucleic acid encoding a non-viral protein selected from the group consisting of immunostimulatory factors, antibodies, and checkpoint blocking peptides, wherein the at least one nucleic acid is operably linked to a generic, or, a tumor-specific promoter. Examples of generic promoters include constitutive promoters such as SV40, CMV, UBC, EF1alpha, PGK and CAG.

12. The recombinant herpes virus according to any one of embodiments, 1, 2, 3, 4, 5, 6, 7, 8, 9, or 11, wherein the non-viral protein is selected from the group consisting of IL12, IL15, IL15 receptor alpha subunit.

13. The recombinant herpes virus according to any one of embodiments 11 or 12, wherein the promoter is a tumor-specific CXCR4 promoter.

14. The recombinant herpes virus according to any one of embodiments 1, 2, 3, 4, 5, 6, 7, 8, 9, 10, 11, 12, or 13 having a nucleic acid sequence encoding a glycoprotein with enhanced fusogenicity (as compared to a similar wild-type virus). Examples include a wide variety of transgenes (e.g., a fusogenic glycoprotein from Gibbon Ape Leukemia Virus “GALV”), and/or mutations which enhance HSV fusion, including for example, a truncations or mutations in glycoprotein B, glycoprotein K, and or UL20. Within a preferred embodiment the nucleic acid sequence encodes a fusogenic form of glycopotein B (e.g., glycoprotein B which is truncated after amino acid 876).

15. The recombinant herpes simplex virus according to embodiment 14, wherein the glycoprotein B can be truncated with a deletion occurring after amino acid 876.

16. The recombinant herpes virus according to any one of embodiments 1, 2, 3, 4, 5, 6, 7, 8, 9, 10, 11, 12, 13, 14, or 15, wherein the oncolytic herpes virus is HSV-1.

Within particularly preferred embodiments of the invention the recombinant herpes virus comprises an oncolytic herpesvirus HSV-1 wherein: a) there is a deletion of a long terminal repeat containing ICPO and ICP34.5 genes; b) replacement of a native ICP27 promoter with a CEA promoter; c) insertion of binding sites for miR-143 and miR-124 in the ICP34.5 3′ UTR; d) deletion of a portion of the 3′ end of glycoprotein B coding region (e.g., a 84 bp deletion); and e) insertion of an expression cassette which can express L-12, IL-15, and IL-15Ra under the control of a CXCR4 promoter.

17. A method for inhibiting or lysing tumor cells, comprising providing a therapeutically effective amount of recombinant herpes virus according to any one of embodiments 1 to 16.

18. A therapeutic composition comprising the recombinant herpes virus according to any one of embodiments 1 to 16 and a pharmaceutically acceptable carrier. Within preferred embodiments the herpes virus is VG2025 as set forth in Table 1 above, or more particularly, hVG2025.

19. A method for treating cancer in a subject suffering therefrom, comprising the step of administering a therapeutically effective amount of the composition of embodiment 18.

20. The method according to embodiment 19 wherein said cancer expresses a high level of a biomarker, the promoter of which is used to drive ICP4 and/or ICP27 genes according to one of the preceding embodiments. Within other embodiments, the cancer expresses a high level of a biomarker such as, for example, hCEA. Within related embodiments the subject is tested for expression of high levels of hCEA (e.g., greater than 2.5 ng/ml) prior to administration of the herpes virus as described herein. Levels of hCEA greater than 10 ng/ml, or even greater than 20 ng/ml indicate significant progression of cancer.

Within certain embodiments, the cancer is selected from the group consisting of cancers of the cervix, esophagus, lung, colorectum, liver stomach, cholangiocarcinoma and pancreas. Within other embodiments the cancer is selected from the group consisting of breast and prostate tumors, and glioblastomas. Within other embodiments the cancer is a leukemia or a lymphoma. Within other embodiments the cancer is an acute myeloid leukemia (AML) or a B cell lymphoma. Within other embodiments, the cancer is a surface injectable tumor. Within yet other embodiments the cancer expresses a high level of CEA.

21. The method according to embodiment 19 wherein said step of administering a therapeutically effective amount of the composition of embodiment 18 comprises intravenous or intratumoral administration. Within preferred embodiments the compositions can be administered at a dosage ranging from about 10⁴ pfu to about 10¹⁰ pfu. Within further embodiments, the dosage can range from about 10⁶ pfu to about 10⁷ pfu, or from about 10⁷ pfu to about 10⁸ pfu, or from about 10⁸ pfu to 10⁹ pfu, and may be administered as a single dose or as multiple doses spread out over time. Doses may also be administered as described in more detail above.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

1. A recombinant herpes virus comprising a modified oncolytic herpes virus genome, wherein the modified herpes virus genome comprises at least one miRNA target sequence operably linked to a first copy of an ICP34.5 gene, and a second copy of the ICP34.5 gene comprises an inactivating mutation, wherein the mutation is deletion of at least one terminal repeat long region of the viral genome.

2. (canceled)

3. The recombinant herpes virus of claim 1, wherein the herpes virus is a herpes simplex virus, and further comprising from two to ten miRNA target sequences operably linked to the first copy of the ICP34.5 gene, wherein the miRNA target sequences are inserted into a 3′ untranslated region of the first copy of the ICP34.5 gene.

4. (canceled)

5. The recombinant herpes simplex virus of claim 3, wherein the from two to ten miRNA target sequences bind at least two different miRNAs, wherein the miRNA target sequence targets an miRNA selected from the group consisting of miR-124, miR-124*, and miR-143.

6. (canceled)

7. The recombinant herpes virus of claim 1, wherein the herpes virus is herpes simplex virus and the modified herpes virus genome comprises additional mutations or modifications in viral genes ICP4 and/or ICP27.

8. The recombinant herpes virus of claim 1, wherein said virus is modified by replacing a native viral promoter with a tumor specific promoter.

9. The recombinant herpes virus of claim 1, wherein the herpes virus is a herpes simplex virus and the modification is replacement of the entire promoter-regulatory region of ICP 4 or ICP27 with a tumor specific promoter.

10. The recombinant herpes simplex virus of claim 9, wherein the ICP27 promoter is replaced with a hCEA promoter.

11. The recombinant herpes virus of claim 1, further comprising at least one nucleic acid encoding a non-viral protein selected from the group consisting of immunostimulatory factors, antibodies, and checkpoint blocking peptides, wherein the at least one nucleic acid is operably linked to a generic or a tumor-specific promoter.

12. The recombinant herpes virus of claim 11, wherein the non-viral protein is selected from the group consisting of IL12, IL15, IL15 receptor alpha subunit.

13. The recombinant herpes virus of claim 12, wherein the tumor-specific promoter is CXCR4 promoter.

14. The recombinant herpes virus of claim 1, wherein the herpes virus is herpes simplex virus, and further comprising a nucleic acid sequence encoding a fusogenic form of glycoprotein B, wherein the glycoprotein B can be truncated with a deletion occurring after amino acid 876.

15. (canceled)

16. The recombinant herpes virus of claim 1, wherein the oncolytic herpes virus is HSV-1.

17. A method for inhibiting tumor cells, comprising providing a therapeutically effective amount of recombinant herpes virus according to claim 1.

18. A therapeutic composition comprising the recombinant herpes virus according to claim 1 and a pharmaceutically acceptable carrier.

19. A method for treating cancer in a subject suffering therefrom, comprising the step of administering a therapeutically effective amount of the composition of claim 18.

20. The method according to claim 19 wherein said cancer expresses a high level of a biomarker.

21. The method according to claim 19 wherein said cancer is selected from the group consisting of cancers of the cervix, esophagus, lung, colorectum, liver, stomach, cholangiocarcinoma and pancreas.

22. The method according to claim 19 wherein said cancer is a leukemia or a lymphoma.

23. The method according to claim 22 wherein said cancer is an acute myeloid leukemia (AML) or a B cell lymphoma.

24. The method according to claim 19 wherein said step of administering a therapeutically effective amount of the composition comprises intravenous (i.v.) or intratumoral administration.
